SEQUENCE LISTING (14 sequences)

SEQ ID NO: 1 - RNA, 19 nt, synthetic construct
  misc_feature 1..19: note = synthetic Polynucleotide
  source 1..19: mol_type = other RNA
  modified_base 4: mod_base = OTHER; note = Uracil
  modified_base 7: mod_base = OTHER; note = Uracil
gcgtactccggcagcgaaa

SEQ ID NO: 2 - RNA, 19 nt, synthetic construct
  misc_feature 1..19: note = synthetic Polynucleotide
  source 1..19: mol_type = other RNA
  modified_base 3: mod_base = OTHER; note = Uracil
  modified_base 4: mod_base = OTHER; note = Uracil
  modified_base 11: mod_base = OTHER; note = Uracil
  modified_base 19: mod_base = OTHER; note = Uracil
gcttcaaggatgaccgcat

SEQ ID NO: 3 - DNA, 16 bp, synthetic construct
  misc_feature 1..16: note = synthetic Polynucleotide
  source 1..16: mol_type = other DNA
  misc_feature 1..3: note = locked nucleic acids
  misc_feature 4..13: note = DNA
  misc_feature 14..16: note = locked nucleic acids
aacacacgccgtcaaa

SEQ ID NO: 4 - DNA, 16 bp, synthetic construct
  misc_feature 1..16: note = synthetic Polynucleotide
  source 1..16: mol_type = other DNA
  misc_feature 1..3: note = locked nucleic acids
  misc_feature 14..16: note = locked nucleic acids
  misc_feature 4..13: note = DNA
acgtaggttaagtgtg

SEQ ID NO: 5 - DNA, 16 bp, synthetic construct
  misc_feature 1..16: note = synthetic Polynucleotide
  source 1..16: mol_type = other DNA
  misc_feature 1..3: note = locked nucleic acids
  misc_feature 4..13: note = DNA
  misc_feature 14..16: note = locked nucleic acids
actagagcaagaggac

SEQ ID NO: 6 - DNA, 20 bp, synthetic construct
  source 1..20: mol_type = other DNA
aaaagcatgtcgtcctgccg

SEQ ID NO: 7 - DNA, 20 bp, synthetic construct
  source 1..20: mol_type = other DNA
gggagaccctgacatcgact

SEQ ID NO: 8 - DNA, 52 bp, synthetic construct
  misc_feature 1..52: note = synthetic Polynucleotide
  source 1..52: mol_type = other DNA
  misc_feature 22..31: note = DNA
  misc_feature 1..21: note = locked nucleic acids
  misc_feature 32..52: note = locked nucleic acids
gacattcacatccataagaagttggatccaacttcttatggatgtgaatgtc

SEQ ID NO: 9 - DNA, 52 bp, synthetic construct
  misc_feature 1..52: note = synthetic Polynucleotide
  source 1..52: mol_type = other DNA
  misc_feature 1..21: note = locked nucleic acids
  misc_feature 22..31: note = DNA
  misc_feature 32..52: note = locked nucleic acids
gcaacatggagtttgtgcgcattggatccaatgcgcacaaactccatgttgc

SEQ ID NO: 10 - DNA, 20 bp, synthetic construct
  source 1..20: mol_type = other DNA
gcggtgtgcacacgtgctga

SEQ ID NO: 11 - DNA, 22 bp, synthetic construct
  misc_feature 1..22: note = synthetic Polynucleotide
  source 1..22: mol_type = other DNA
tctttcagggctccatccgcta

SEQ ID NO: 12 - DNA, 22 bp, synthetic construct
  misc_feature 1..22: note = synthetic Polynucleotide
  source 1..22: mol_type = other DNA
gcgcacaaactccatgttgctg

SEQ ID NO: 13 - DNA, 22 bp, synthetic construct
  misc_feature 1..22: note = synthetic Polynucleotide
  source 1..22: mol_type = other DNA
caccattggcaatgagcggttc

SEQ ID NO: 14 - DNA, 22 bp, synthetic construct
  misc_feature 1..22: note = synthetic Polynucleotide
  source 1..22: mol_type = other DNA
aggtctttgcggatgtccacgt

(19) United States
(12) Patent Application Publication
(10) Pub. No.: US 2024/0011036 A1
(43) Pub. Date: Jan. 11, 2024

(54) PREVENTION OR TREATMENT OF WASTING SYNDROME

(71) Applicant: City of Hope, Duarte, CA (US)

(72) Inventors: Keiichi Itakura, Duarte, CA (US); Takahiro Yamakawa, Duarte, CA (US)

(21) Appl. No.: 18/220,070

(22) Filed: Jul. 10, 2023

Related U.S. Application Data
(60) Provisional application No. 63/388,147, filed on Jul. 11, 2022.

(51) Int. Cl. C12N 15/113; A61K 45/06; A61P 21/00

(52) U.S. Cl. CPC C12N 15/1135; A61K 45/06; A61P 21/00

(57) ABSTRACT

Provided herein are, inter alia, KIAA0930 inhibitors, pharmaceutical compositions, and methods for treating and preventing wasting syndromes, such as cancer cachexia.

### CROSS-REFERENCE TO RELATED APPLICATIONS

This application claims the benefit of priority to U.S. Application No. 63/388,147 filed Jul. 11, 2022, the disclosure of which is incorporated by reference herein in its entirety.

### REFERENCE TO A “SEQUENCE LISTING,” A TABLE, OR A COMPUTER PROGRAM LISTING APPENDIX SUBMITTED AS AN ASCII FILE

The Sequence Listing written in file .xml, created 2023, y bytes is hereby incorporated by reference.

### BACKGROUND

Cancer cachexia (CC) is a multifactorial disease characterized by muscle and fat loss in advanced cancer. CC is observed in 70-90% of pancreatic cancer, 60-70% of gastric cancer, and 40-60% of colorectal cancer, and is thought to be directly responsible for 20-30% of cancer deaths. Patients with CC do not respond to anti-cancer therapies well, resulting in poor prognosis, and impaired quality of life. Therefore the development of therapeutics is an urgent task. Numerous studies showed that pro-inflammatory cytokines act as a major mediator of CC in preclinical models. Therefore, many clinical trials targeting cytokines and its signaling pathways using monoclonal antibodies and kinase inhibitors (e.g. JAK1/2 in IL-6 signaling) have been conducted. However, none of the clinical studies has shown to be effective. Hence, there is an unmet need of developing therapeutics that are based on different targeting approaches for the management and treatment of patients affected by a wasting syndrome. The disclosure is directed to these, as well as other, important ends.

### BRIEF SUMMARY

Provided herein are methods of treating a wasting syndrome in a subject in need thereof. The disclosed methods comprise administering to the subject an effective amount of a KIAA0930 inhibitor. In embodiments, the wasting syndrome is associated with cancer cachexia. Exemplary wasting syndromes include, but are not limited to, weight loss, fat loss, muscle atrophy, anorexia, asthenia, and anemia. In embodiments, the subject does not respond to anti-cancer therapies.

In embodiments, the KIAA0930 inhibitor is a short-hairpin RNA (shRNA), a small interference RNA (siRNA), a piwi-interacting RNA (piRNA), a microRNA (miRNA), an antisense oligonucleotide such as a GapmeR or a morpholinooligonucleotide, a CRISPR Cas guide RNA (gRNA), or a small molecule compound.

Provided herein are KIAA0930 inhibitors and pharmaceutical compositions comprising KIAA0930 inhibitors. The disclosed pharmaceutical compositions comprise a KIAA0930 inhibitor in an amount effective to treat a wasting syndrome in a subject in need thereof.

These and other embodiments of the disclosure are described in detail herein.

### BRIEF DESCRIPTION OF THE DRAWINGS

FIGS. 1A-1J illustrate the effect of KIAA0930 siRNA on KIAA0930 mRNA expression. KIAA0930 siRNA1 and KIAA0930 siRNA2 reduce KIAA0930 mRNA expression. After 1 day, cells were cultured for 3 days and total RNA was extracted. mRNA was measured using real-time RT-PCR. These data are representative of two independent experiments. The y-axis is KIAA0930 per (3-Actin.

FIGS. 2A-2J show that KIAA0930 GapmeR1-3 reduces KIAA0930 mRNA expression. Cells were transfected with GapmeR1-2, or nontargeted GapmeR (control). After 1 day, the cells were cultured for 3 days and total RNA was extracted. mRNA was measured using real-time RT-PCR. The data are representative of two independent experiments. The y-axis is KIAA0930 per (3-Actin.

FIGS. 3A-3I show that KIAA0930 shRNA1 and KIAA0930 shRNA2 reduce KIAA0930 mRNA expression. Cells stably expressing shRNA1, shRNA2, or nontargeted shRNA were cultured for 3 days and total RNA was extracted. mRNA was measured using real-time RT-PCR. Results are shown as mean±S.E. from three to eleven independent experiments. **p<0.01 vs. Control. The y-axis is KIAA0930 per (3-Actin.

FIGS. 4A-4B show that KIAA0930 guideRNA reduces KIAA0930 protein in HCT116 cells. FIG. 4A: Cas9-overexpressing HCT116 cells were transfected with sgRNA1, sgRNA2, or nontargeted sgRNA (control sgRNA). After 1 day, the cells were cultured for 3 days and whole cell extract was prepared. KIAA0930 protein was measured using Western Blotting. FIG. 4B: Cas9 and gRNA3-expressing HCT116 cells were cultured for 3 days and a whole cell extract was prepared. Lysates from control shRNA, shRNA1 and shRNA2-expressing cells served as positive and negative controls, respectively. Arrow shows KIAA0930 protein. The data are representative of two independent experiments.

FIGS. 5A-5J show that conditioned medium from KIAA0930 siRNA1 and KIAA0930 siRNA 2-treated cells ameliorates myotube atrophy. The data are representative of two independent experiments. Mean±S.E. (n=44 myotubes) representative from two independent experiments. **p<0.01 vs. NTsiRNA CM § p<0.01 vs. NCM. NS: Not significant. The y-axis is myotube diameter in μm.

FIGS. 6A-6J show that conditioned medium from KIAA0930 GapmeR1-3-treated cells ameliorates myotube atrophy. Mean±S.E. (n=44 myotubes) representative from two independent experiments. *p<0.05, **p<0.01 vs. Control CM § p<0.01 vs. NCM. NS: Not significant.

FIGS. 7A-7I show that conditioned medium from KIAA0930 shRNA1, KIAA0930 shRNA2-expressing cells ameliorates myotube atrophy. Mean±S.E. (n=39-46 myotubes) representative from three independent experiments. **p<0.01, *p<0.05, vs. Control CM §§ p<0.01 vs. NCM. NS: Not significant.

FIGS. 8A-8B show that conditioned medium from KIAA0930 guideRNA1-3-treated HCT116-Cas9 cells ameliorates myotube atrophy. Mean±S.E. (n=44 myotubes) representative from two independent experiments. **p<0.01 vs. Control CM § p<0.01 vs. NCM. NS: Not significant.

FIGS. 9A-9EE illustrate the cytokine contents in CM from KIAA0930 knockdown cancer cells. FIGS. 9A-9C: MCP-1. Capan-2, Mia PaCa-2, HCT116, HT29, and MKN45 cells do not produce MCP-1. FIGS. 9D-9K: TGFB1. FIGS. 9L-9S: TGFB2. FIGS. 9T-9W: IL-6. PANC-1, HCT116, HT29, and MKN45 cells do not produce IL-6. FIGS. 9X-9EE: IL-8. The data are shown as mean±S.E. from three independent experiments. **p<0.01, *p<0.05 vs. Control.

FIGS. 10A-10E show that KIAA0930 knockdown ameliorates muscle atrophy in PANC-1 orthotopic xenograft model. FIGS. 10A-10C: Body weight, tumor and TA weight in PBS-, PANC-1 expressing shRNA1, 2-inoculated mice. FIG. 10D: Representative images of TA muscle sections stained with H-E. FIG. 10E: The quantification of cross section area in TA. The data are shown as mean±S.E. (n=5-6). **p<0.01 vs. Control group, §§ p<0.01 vs. PBS group.

FIGS. 11A-1111 show conditioned medium from KIAA0930 siRNA-treated hTERT-HPaSteC, HHSteC and CCD-18co cells, followed by addition of cancer cell CM in the culture, ameliorates myotube atrophy. These data are representative of two independent experiments. Results are shown as representative from two independent experiments (Mean±S.E., n=44 myotubes), **p<0.01 vs. NTsiRNA, § p<0.01 vs. No cells, NS: Not significant. Graph on the right in FIGS. 11A-11H shows KIAA0930 mRNA expression normalized to (3-actin.

FIGS. 12A-12B are a schematic diagram from the results shown in FIGS. 11A-11H.

### DETAILED DESCRIPTION

### Definitions

Unless defined otherwise, technical and scientific terms used herein have the same meaning as commonly understood by a person of ordinary skill in the art. See, e.g., Singleton et al., Dictionary of Microbiology and Molecular Biology, 2nd ed., J. Wiley & Sons (New York, NY 1994); Sambrook et al., Molecular Cloning, A Laboratory Manual, Cold Springs Harbor Press (Cold Springs Harbor, N Y 1989). Any methods, devices and materials similar or equivalent to those described herein can be used in the practice of this disclosure. The following definitions are provided to facilitate understanding of certain terms used frequently herein and are not meant to limit the scope of the present disclosure.

It must be noted that as used herein and in the appended claims, the singular forms “a,” “an,” and “the” include plural references unless the context clearly dictates otherwise.

The term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about means the specified value.

The term “KIAA0930” is used in accordance with its plain ordinary meaning and refers to an uncharacterized protein, also known as “chromosome 22 open reading frame 9” or “C22orf9,” which is encoded by the C22orf9 gene. The term “KIAA0930” as used herein includes all isoforms of KIAA0930 (Q6ICG6-1˜3). In embodiments, the isoform of the KIAA0930 protein is Q6ICG6-1, and the KIAA0930 protein contains 404 amino acids. In embodiments, the isoform of the KIAA0930 protein is Q6ICG6-2, and the KIAA0930 protein contains 407 amino acids. In embodiments, the isoform of the KIAA0930 protein is Q6ICG6-3, and the KIAA0930 protein contains 370 amino acids. In embodiments, the KIAA0930 protein is expressed in all tissues. In embodiments, the KIAA0930 protein is highly expressed in adult and fetal brain. In embodiments, the KIAA0930 protein is highly expressed in the corpus callosum and the subthalamic nucleus in the brain. In embodiments, the KIAA0930 protein is highly expressed in the spinal cord.

The term “wasting syndrome” or “cachexia” refers to a condition which denotes an involuntary loss of more than 10% of body weight (especially muscle mass), plus at least 30 days of either diarrhea or weakness and fever. Wasting syndrome occurs because of the depletion of adipose tissue and muscle mass in people who are not trying to lose weight. It causes disproportionate muscle wasting, weakness, fatigue, and loss of appetite in affected individuals. The word “cachexia” originates from two Greek terms “kakos” which means “bad” and “hexis” which means “condition.” Multiple cytokines have been proposed to mediate cachexia including TNF-α, IL-1, IL-6, IL-11, IFN-γ, and leukemia inhibitory factor (LIF). However, the exact mechanism of cytokine-induced cachexia is unknown. Cachexia is seen in a number of patients with conditions such as AIDS, cancer, celiac disease, rheumatoid arthritis, multiple sclerosis, congestive heart failure, tuberculosis, mercury poisoning, severe sepsis, and malabsorption. Weight loss in cachexia involves loss of equal amounts of fat and muscle. Hence, for a given percentage of weight loss, a cachectic person loses more muscle than a starving person. Unlike cases of simple starvation, the weight loss or change in body composition in the case of cachexia is not reversible by ensuring adequate calorie ingestion. This is because of profound metabolic changes taking place in cachexia, which lead to a higher basal rate of energy expenditure, as well as to the increased degradation of fat and muscle. The altered body composition of the patient at presentation helps to differentiate cachexia from other syndromes such as anorexia causing weight loss. Cachexia can significantly compromise quality of life and functional status is associated with poor outcomes However, anorexia may be a contributing factor to the muscle wasting seen in patients with cachexia. This is because the loss of appetite and reduced intake of food interferes with the psychological and physical quality of life of the patient.

The term “muscle atrophy” refers to the thinning or loss of muscle tissue. Muscle atrophy can occur due to malnutrition, age, genetics, lack of physical activity or certain medical conditions. Disuse (physiologic) atrophy occurs when muscles are not used enough. Neurogenic atrophy occurs due to nerve problems or diseases. The most obvious sign of muscle atrophy is reduced muscle mass. Other signs of muscle atrophy may include weakness, numbness, trouble walking or balancing, and difficulty swallowing or speaking. Disuse causes rapid muscle atrophy and often occurs during injury or illness that requires immobilization of a limb or bed rest. Depending on the duration of disuse and the health of the individual, this may be fully reversed with activity. Malnutrition first causes fat loss but may progress to muscle atrophy in prolonged starvation and can be reversed with nutritional therapy. Cachexia is a wasting syndrome caused by an underlying disease such as cancer that causes dramatic muscle atrophy and cannot be completely reversed with nutritional therapy. Sarcopenia is an age-related muscle atrophy and can be slowed by exercise. Diseases of the muscles such as muscular dystrophy or myopathies can cause atrophy, as well as damage to the nervous system. Thus, muscle atrophy is usually a symptom of a disease rather than a disease itself. Muscle atrophy results from an imbalance between protein synthesis and protein degradation, although the mechanisms are incompletely understood and are variable depending on the cause. Muscle loss can be quantified with advanced imaging studies but this is not frequently pursued. Treatment depends on the underlying cause but will often include exercise and adequate nutrition. Anabolic agents may have some efficacy but are not often used due to side effects. There are multiple treatments and supplements under investigation but there are currently limited treatment options in clinical practice. Given the implications of muscle atrophy and limited treatment options, minimizing immobility is critical in injury or illness.

The term “weight loss,” “unexplained weight loss,” and “involuntary weight loss” refers to an unintentional loss of 5% or more of bodyweight within a period of six months to one year, which occurs without changing diet or exercise routine. Unexplained weight loss may occur as a result of a stressful event, malnutrition, a health condition, or any combination thereof. Some causes of unintentional weight loss include mental health conditions, such as depression, anxiety, eating disorders, and obsessive compulsive disorder, digestive problems due to coeliac disease or irritable bowel syndromes, or other health conditions, such as an overeactive thyroid, diabetes, heart failure, or cancer.

The term “fat loss,” “unexplained fat loss,” and “involuntary fat loss” refers to an unintentional loss of adipose tissue. Fat loss occurs in both visceral and subcutaneous depots. Increased lipolysis and fat oxidation, decreased lipogenesis, impaired lipid deposition and adipogenesis, as well as browning of white adipose tissue may underlie adipose atrophy in cancer. Adipose tissue, a main player in cancer cachexia, is an essential metabolic and secretory organ consisting of both white adipose tissue (WAT) and brown adipose tissue. Its secretory products, including adipokines and cytokines, affect a wide variety of central and peripheral organs, such as the skeletal muscle, brain, pancreas, and liver. A combination of metabolic alterations and systemic inflammation dysregulation of both anti-inflammatory and proinflammatory modulators contribute toward adipose tissue wasting in cancer cachexia. Growing evidence suggests that, during cancer cachexia, WAT undergoes a browning process, resulting in increased lipid mobilization and energy expenditure. Adipose tissue may become inflamed in cancer, with consequent adipose tissue dysfunction. Loss of adipose tissue has been attributed to increased adipocyte lipolysis, systemic inflammation, and apoptosis or reduced lipogenesis.

The term “anorexia nervosa” or “anorexia” refers to an eating disorder characterized by low weight, food restrictions, body image disturbance, fear of gaining weight, and an overpowering desire to be thin. Anorexia is a term of Greek origin: an- ( [symbol] ν-, prefix denoting negation) and orexis ( [symbol] , “appetite”), translating literally to “a loss of appetite,” which is of non-organic nature of the disorder. Individuals with anorexia nervosa commonly see themselves as being overweight, despite the fact that they are often underweight. Individuals with anorexia nervosa also often deny that they have a problem with low weight. Medical complications may include osteoporosis, infertility, and heart damage, among others. The cause of anorexia is currently unknown. Anorexia often begins following a major life-change or stress-inducing event. The severity of the disease is based on body mass index (BMI) in adults with mild disease having a BMI of greater than 17, moderate a BMI of 16 to 17, severe a BMI of 15 to 16, and extreme a BMI less than 15. Treatment of anorexia involves restoring the patient back to a healthy weight, treating their underlying psychological problems, and addressing behaviors that promote the problem.

The term “asthenia” refers to weackness and lack of energy and strength. General asthenia occurs in many chronic wasting diseases, such as anemia and cancer, and is most marked in diseases of the adrenal gland. Asthenia may be limited to certain organs or systems of organs, as in asthenopia, characterized by ready fatigability of vision, or in myasthenia gravis, in which there is progressive increase in the fatigability of the muscular system. Neurocirculatory asthenia is a clinical syndrome characterized by breathing difficulties, heart palpitations, shortness of breath or dizziness, and insomnia.

The term “anemia” refers to a condition that develops when the blood produces a lower-than-normal amount of healthy red blood cells, such that the body does not get enough oxygen-rich blood. The lack of oxygen causes a person to feel tired or weak. Accompanying symptoms may include shortness of breath, dizziness, headaches, or an irregular heartbeat. Different types of anemia have different causes. They include iron deficiency anemia; vitamin deficiency anemia, which is caused by the lack of folate and vitamin B-12 tin the diet; anemia of inflammation, which occurs when certain diseases, such as cancer, HIV/AIDS, rheumatoid arthritis, kidney disease, Crohn's disease and other acute or chronic inflammatory diseases interfere with the production of red blood cells; aplastic anemia, a rare, life-threatening condition caused by infections, autoimmune diseases and exposure to toxic chemicals; anemias associated with bone marrow disease; hemolytic anemias, which occur when red blood cells are destroyed faster than bone marrow can replace them; and sickle cell anemia, which is caused by a defective form of hemoglobin that forces red blood cells to assume an abnormal crescent (sickle) shape. These irregular blood cells die prematurely, resulting in a chronic shortage of red blood cells. Chronic conditions, such as cancer and kidney failure, can lead to a shortage of red blood cells.

“Nucleic acid” refers to deoxyribonucleotides or ribonucleotides and polymers thereof in either single- or double-stranded forms, and complements thereof. The term “polynucleotide” refers to a linear sequence of nucleotides. The term “nucleotide” typically refers to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA (including siRNA), and hybrid molecules having mixtures of single and double stranded DNA and RNA. Nucleic acid as used herein also refers to nucleic acids that have the same basic chemical structure as a naturally occurring nucleic acid. Such analogues have modified sugars and/or modified ring substituents, but retain the same basic chemical structure as the naturally occurring nucleic acid. A nucleic acid mimetic refers to chemical compounds that have a structure that is different from the general chemical structure of a nucleic acid, but that functions in a manner similar to a naturally occurring nucleic acid. Examples of such analogues include, without limitation, phosphorothioates, phosphoramidates, methyl phosphonates, chiral-methyl phosphonates, 2-O-methyl ribonucleotides, and peptide-nucleic acids (PNAs).

The term “nucleotide” typically refers to a compound containing a nucleoside or a nucleoside analogue and at least one phosphate group or a modified phosphate group linked to it by a covalent bond. Exemplary covalent bonds include, without limitation, an ester bond between the 3′, 2′ or 5′ hydroxyl group of a nucleoside and a phosphate group.

The term “nucleoside” refers to a compound containing a sugar part and a nucleobase, e.g., a pyrimidine or purine base. Exemplary sugars include, without limitation, ribose, 2-deoxyribose, arabinose and the like. Exemplary nucleobases include, without limitation, thymine, uracil, cytosine, adenine, guanine.

The term “nucleoside analogue” may refer to a nucleoside any part of which is replaced by a chemical group of any nature. Exemplary nucleoside analogues include, without limitation, 2′-substituted nucleosides such as 2′-fluoro, 2-deoxy, 2′-O-methyl, 2′-O—P-methoxyethyl, 2′-O-allylriboribonucleosides, 2′-amino, locked nucleic acid (LNA) monomers and the like. The term “nucleoside analogue” may also refer to a nucleoside in which the sugar or base part is modified, e.g. with a non-naturally occurring modification. Exemplary nucleoside analogues in which the sugar part is replaced with another cyclic structure include, without limitation, monomeric units of morpholinos (PMO) and tricyclo-DNA. Exemplary nucleoside analogues in which the sugar part is replaced with an acyclic structure include, without limitation, monomeric units of peptide nucleic acids (PNA) and glycerol nucleic acids (GNA). Suitably, nucleoside analogues may include nucleoside analogues in which the sugar part is replaced by a morpholine ring.

Nucleoside analogues may include deoxyadenosine analogues, adenosine analogues, deoxycytidine analogues, cytidine analogues, deoxyguanosine analogues, guanosine analogues, thymidine analogues, 5-methyluridine analogues, deoxyuridine analogues, or uridine analogues. Examples of deoxyadenosine analogues include didanosine (2′, 3′-dideoxyinosine) and vidarabine (9-D-arabinofuranosyladenine), fludarabine, pentostatin, cladribine. Examples of adenosine analogues include BCX4430 (Immucillin-A). Examples of cytidine analogues include gemcitabine, 5-aza-2′-deoxycytidine, cytarabine. Examples of deoxycytidine analogues include cytarabine, emtricitabine, lamivudine, zalcitabine. Examples of guanosine and deoxyguanosine analogues include abacavir, acyclovir, entecavir. Examples of thymidine and 5-methyluridine analogues include stavudine, telbivudine, zidovudine. Examples of deoxyuridine analogues include idoxuridine and trifluridine.

The term “gene” means the segment of DNA involved in producing a protein; it includes regions preceding and following the coding region (leader and trailer) as well as intervening sequences (introns) between individual coding segments (exons). The leader, the trailer, as well as the introns, include regulatory elements that are necessary during the transcription and the translation of a gene. Further, a “protein gene product” is a protein expressed from a particular gene.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell. The level of expression of nucleic acid molecules may be detected by standard PCR or Northern blot methods well known in the art. See, Sambrook et al., 1989 Molecular Cloning: A Laboratory Manual, 18.1-18.88.

Expression of a transfected gene can occur transiently or stably in a cell. During “transient expression” the transfected gene is not transferred to the daughter cell during cell division. Since its expression is restricted to the transfected cell, expression of the gene is lost over time. In contrast, stable expression of a transfected gene can occur when the gene is co-transfected with another gene that confers a selection advantage to the transfected cell. Such a selection advantage may be a resistance towards a certain toxin that is presented to the cell.

The terms “transfection”, “transduction”, “transfecting” or “transducing” are used interchangeably throughout and are defined as a process of introducing a nucleic acid molecule or a protein to a cell. Nucleic acids are introduced to a cell using non-viral or viral-based methods. The nucleic acid molecules may be gene sequences encoding complete proteins or functional portions thereof. Non-viral methods of transfection include any appropriate transfection method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetifection, and electroporation. In some embodiments, the nucleic acid molecules are introduced into a cell using electroporation following standard procedures well known in the art. For viral-based methods of transfection any useful viral vector may be used in the methods described herein. Examples for viral vectors include, but are not limited to retroviral, adenoviral, lentiviral and adeno-associated viral vectors. In some embodiments, the nucleic acid molecules are introduced into a cell using a retroviral vector following standard procedures well known in the art. The terms “transfection” or “transduction” also refer to introducing proteins into a cell from the external environment. Typically, transduction or transfection of a protein relies on attachment of a peptide or protein capable of crossing the cell membrane to the protein of interest. See, e.g., Ford et al. (2001) Gene Therapy 8:1-4 and Prochiantz (2007) Nat. Methods 4:119-20.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., spodoptera) and human cells.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. The term “amino acid analogs” refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that function in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “numbered with reference to” or “corresponding to,” when used in the context of the numbering of a given amino acid or polynucleotide sequence, refer to the numbering of the residues of a specified reference sequence when the given amino acid or polynucleotide sequence is compared to the reference sequence. An amino acid residue in a protein “corresponds” to a given residue when it occupies the same essential structural position within the protein as the given residue. One skilled in the art will immediately recognize the identity and location of residues corresponding to a specific position in a protein in other proteins with different numbering systems. For example, by performing a simple sequence alignment with a protein the identity and location of residues corresponding to specific positions of the protein are identified in other protein sequences aligning to the protein. For example, a selected residue in a selected protein corresponds to glutamic acid at position 138 when the selected residue occupies the same essential spatial or other structural relationship as a glutamic acid at position 138. In some embodiments, where a selected protein is aligned for maximum homology with a protein, the position in the aligned selected protein aligning with glutamic acid 138 is the to correspond to glutamic acid 138. Instead of a primary sequence alignment, a three dimensional structural alignment can also be used, e.g., where the structure of the selected protein is aligned for maximum correspondence with the glutamic acid at position 138, and the overall structures compared. In this case, an amino acid that occupies the same essential position as glutamic acid 138 in the structural model is the glutamic acid 138 residue.

The terms “polypeptide,” “peptide” and “protein” are used interchangeably herein to refer to a polymer of amino acid residues, wherein the polymer may optionally be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure. The following eight groups each contain amino acids that are conservative substitutions for one another: (1) Alanine (A), Glycine (G); (2) Aspartic acid (D), Glutamic acid (E); (3) Asparagine (N), Glutamine (Q); (4) Arginine (R), Lysine (K); (5) Isoleucine (I), Leucine (L), Methionine (M), Valine (V); (6) Phenylalanine (F), Tyrosine (Y), Tryptophan (W); (7) Serine (S), Threonine (T); and (8) Cysteine (C), Methionine (M) (see, e.g., Creighton, Proteins (1984)).

The term “recombinant” when used with reference, for example, to a cell, a nucleic acid, a protein, or a vector, indicates that the cell, nucleic acid, protein or vector has been modified by or is the result of laboratory methods. Thus, for example, recombinant proteins include proteins produced by laboratory methods. Recombinant proteins can include amino acid residues not found within the native (non-recombinant) form of the protein or can be include amino acid residues that have been modified (e.g., labeled).

The terms “identical” or percent “identity,” in the context of two or more nucleic acids or polypeptide sequences, refer to two or more sequences or subsequences that are the same or have a specified percentage of amino acid residues or nucleotides that are the same (i.e., 60% identity, optionally 65%, 70%, 75%, 80%, 85%, 90%, 95%, 98%, or 99% identity over a specified region, e.g., of the entire polypeptide sequences disclosed herein or individual domains of the polypeptides disclosed herein), when compared and aligned for maximum correspondence over a comparison window, or designated region as measured using one of the following sequence comparison algorithms or by manual alignment and visual inspection. Such sequences are then considered to be “substantially identical.” This definition also refers to the complement of a test sequence. Optionally, the identity exists over a region that is at least about 50 nucleotides in length, or more preferably over a region that is 100 to 500 or 1000 or more nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of, e.g., a full length sequence or from 20 to 600, about 50 to about 200, or about 100 to about 150 amino acids or nucleotides in which a sequence may be compared to a reference sequence of the same number of contiguous positions after the two sequences are optimally aligned. Any methods of alignment of sequences for comparison well known in the art are contemplated. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Ausubel et al., Current Protocols in Molecular Biology (1995 supplement)).

Example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

An “antisense nucleic acid” as referred to herein is a nucleic acid (e.g. DNA or RNA molecule) that is complementary to at least a portion of a specific target nucleic acid (e.g. an mRNA translatable into a protein) and is typically capable of reducing transcription of the target nucleic acid (e.g. mRNA from DNA) or reducing the translation or the amount of the target nucleic acid (e.g. mRNA) or altering transcript splicing (e.g. single stranded morpholino oligo). See, e.g., Weintraub, Scientific American, 262:40 (1990). Typically, synthetic antisense nucleic acids (e.g. oligonucleotides) are generally between 15 and 25 bases in length. Thus, antisense nucleic acids are capable of hybridizing to (e.g. selectively hybridizing to) a target nucleic acid (e.g. target mRNA). In embodiments, the antisense nucleic acid hybridizes to the target nucleic acid sequence (e.g. mRNA) under stringent hybridization conditions. In embodiments, the antisense nucleic acid hybridizes to the target nucleic acid (e.g. mRNA) under moderately stringent hybridization conditions. Antisense nucleic acids may comprise naturally occurring nucleotides or modified nucleotides such as, e.g., phosphorothioate, methylphosphonate, and -anomeric sugar-phosphate, backbone modified nucleotides. In the cell, the antisense nucleic acids may hybridize to the corresponding mRNA, forming a double-stranded molecule. The antisense nucleic acids interfere with the translation of the mRNA, since the cell will not translate an mRNA that is double-stranded. The use of antisense methods to inhibit the in vitro translation of genes is well known in the art (Marcus-Sakura, Anal. Biochem. 172:289, (1988)). Further, antisense molecules which bind directly to the DNA may be used. Antisense nucleic acids may be single or double stranded nucleic acids. Non-limiting examples of antisense nucleic acids include siRNAs (including their derivatives or pre-cursors, such as nucleotide analogues), short hairpin RNAs (shRNA), micro RNAs (miRNA), saRNAs (small activating RNAs) and small nucleolar RNAs (snoRNA) or certain of their derivatives or pre-cursors.

A “siRNA,” “small interfering RNA,” “small RNA,” or “RNAi” as provided herein, refers to a nucleic acid that forms a double stranded RNA, which double stranded RNA has the ability to reduce or inhibit expression of a gene or target gene when present in the same cell as the gene or target gene. The complementary portions of the nucleic acid that hybridize to form the double stranded molecule typically have substantial or complete identity. In embodiments, a siRNA or RNAi is a nucleic acid that has substantial or complete identity to a target gene and forms a double stranded siRNA. In embodiments, the siRNA inhibits gene expression by interacting with a complementary cellular mRNA thereby interfering with the expression of the complementary mRNA. Typically, the nucleic acid is at least about 15-50 nucleotides in length (e.g., each complementary sequence of the double stranded siRNA is 15-50 nucleotides in length, and the double stranded siRNA is about 15-50 base pairs in length). In embodiments, the length is 20-30 base nucleotides, preferably about 20-25 or about 24-29 nucleotides in length, e.g., 20, 21, 22, 23, 24, 25, 26, 27, 28, 29, or 30 nucleotides in length.

A “saRNA,” or “small activating RNA” as provided herein refers to a nucleic acid that forms a double stranded RNA, which double stranded RNA has the ability to increase or activate expression of a gene or target gene when present in the same cell as the gene or target gene. The complementary portions of the nucleic acid that hybridize to form the double stranded molecule typically have substantial or complete identity. In embodiments, a saRNA is a nucleic acid that has substantial or complete identity to a target gene and forms a double stranded saRNA. Typically, the nucleic acid is at least about 15-50 nucleotides in length (e.g., each complementary sequence of the double stranded saRNA is 15-50 nucleotides in length, and the double stranded saRNA is about 15-base pairs in length). In embodiments, the length is 20-30 base nucleotides, preferably about 20- or about 24-29 nucleotides in length, e.g., 20, 21, 22, 23, 24, 25, 26, 27, 28, 29, or 30 nucleotides in length.

A “shRNA,” “short hairpin RNA,” or “small hairpin RNA” as provided herein refers to an RNA molecule including a hairpin turn that has the ability to reduce or inhibit expression of a target gene or target nucleic acid when expressed in the same cell as the target gene or target nucleic acid. shRNA expression in a cell may be accomplished by delivery of the shRNA the cell using a plasmid or vector. Typically, the shRNA is cleaved by an enzyme (i.e. Dicer) to produce an siRNA product. The siRNA may then associate with RISC, thereby allowing target recognition.

A “PIWI-interacting RNA” or “piRNA” refers to a type of small non-coding RNA (sncRNA), which is 26-31 nucleotides in length and binds to PIWI proteins. Their features include the following characteristics: piRNAs are independent of the Dicer enzyme and are produced by a single-stranded precursor. The majority of piRNA clusters in somatic cells are unidirectional, whereas the majority of germline piRNA clusters are dual-stranded. Most mature primary piRNAs contain uridine at the 5′ end, and the 3′ ends of piRNAs are uniquely methylated 2-OH structures. piRNAs are unevenly distributed among various genomic sequences, including exons, introns, and repeat sequences. piRNAs are derived from transposons and from flanking genomic sequences. piRNAs are not degraded in circulation and are stably expressed in body fluids.

PIWI proteins are mainly expressed in the germline and human tumors. The human PIWI protein subfamily consists of PIWIL1, PIWIL2, PIWIL3 and PIWIL4. piRNAs are essential in many stages of spermatogenesis, and PIWIs are necessary to maintain the function of reproductive system stem cells. piRNAs interact with PIWI subfamily proteins, resulting in the development of the piRNA-induced silencing complex (piRISC), which detects and silences complementary sequences at the transcriptional (TGS) and post-transcriptional (PTGS) levels. The absence of piRNAs can lead to pathogenic effects in the reproductive system, such as birth defects and infertility piRNAs are thought to be essential regulators for germline preservation, and they can also influence gene expression in somatic cells. Dysregulation of piRNAs can both promote and repress the emergence and progression of human cancers through DNA methylation, transcriptional silencing, mRNA turnover, and translational control. piRNAs control the expression of essential genes and pathways associated with digestive cancer progression and have been reported as possible biomarkers for the diagnosis and treatment of digestive cancer.

A “gapmeR” as provided herein refers to a short DNA anti sense oligonucleotide flanked by strands of RNA mimics. The mimics are typically composed of locked nucleic acids (LNAs), 2′-OMe, or 2′-F modified bases. LNA sequences are RNA analogues “locked” into an ideal Watson-Crick base pairing conformation. LNAs, 2′-OMe, or 2′-F modified bases are chemical analogs of natural RNA nucleic acids and allow for an increase in nuclease resistance, reduced immunogenicity, and a decrease in toxicity. Gapmers can also have a high binding affinity to the target mRNA. This high binding affinity reduces off-target effects, non-specific binding, and unwanted gene silencing. GapmeRs often utilize nucleotides modified with phosphorothioate (PS) groups. In humans, the gapmer DNA-mRNA duplex is degraded by RNase H. The degradation of the mRNA prevents protein synthesis. GapmeRs are designed to hybridize to a target RNA sequence and silence the gene through the induction of RNase H cleavage. Binding of the gapmer to the target has a higher affinity due to the modified RNA flanking regions, as well as resistance to degradation by nucleases. GapmeRs are currently being developed as therapeutics for a variety of cancers, viruses, and other chronic genetic disorders.

A “morpholinooligonucleotide,” “morpholino oligonucleotide,” “mporpholino,” “morpholino oligomer,” or “morpholino oligo” as used herein refers to synthetic antisense oligonucleotide of about 25 nucleotides in length designed to bind and block the translation initiation complex of messenger RNA (mRNA) sequences. Morpholinooligonucleotides contain DNA bases attached to a backbone of methylenemorpholine rings linked through phosphorodiamidate groups. Morpholinos act by “steric blocking”, binding to a target sequence within an RNA molecule, thereby inhibiting molecules that might otherwise interact with the RNA. By sterically blocking the translation initiation complex, morpholinos can knock down expression of many target sequences. Unlike many antisense types (e.g. siRNA, phosphorothioates), morpholinos generally do not cause degradation of their RNA targets; instead, they block the biological activity of the target RNA until that RNA is degraded naturally, which releases the morpholino. In addition, morpholinos can be used to modify and control normal splicing events by blocking sites involved in splicing pre-mRNA. Morpholinos must be actively delivered into most cells by a variety of methods, including scrape-loading of adherent cells, electroporation, and microinjection. A Morpholino oligo is radically different from natural nucleic acids, with methylenemorpholine rings replacing the ribose or deoxyribose sugar moieties and non-ionic phosphorodiamidate linkages replacing the anionic phosphates of DNA and RNA. Each morpholine ring suitably positions one of the standard DNA bases (A,C,G,T) for pairing, so that a 25-base morpholino oligo strongly and specifically binds to its complementary 25-base target site in a strand of RNA via Watson-Crick pairing. Because the uncharged backbone of the morpholino oligo is not recognized by enzymes, it is completely stable to nucleases.

A “guide RNA” or “gRNA” as provided herein refers to any polynucleotide sequence having sufficient complementarity with a target polynucleotide sequence to hybridize with the target sequence and direct sequence-specific binding of a CRISPR complex to the target sequence. In aspects, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more.

In embodiments, the polynucleotide (e.g., gRNA) is a single-stranded ribonucleic acid. In aspects, the polynucleotide (e.g., gRNA) is from about 10 to about 200 nucleic acid residues in length. In aspects, the polynucleotide (e.g., gRNA) is from about 50 to about 150 nucleic acid residues in length. In aspects, the polynucleotide (e.g., gRNA) is from about 80 to about 140 nucleic acid residues in length. In aspects, the polynucleotide (e.g., gRNA) is from about 90 to about 130 nucleic acid residues in length. In aspects, the polynucleotide (e.g., gRNA) is from about 100 to about 120 nucleic acid residues in length. In aspects, the length of the polynucleotide (e.g., gRNA) is about 113 nucleic acid residues in length.

In general, a guide sequence (i.e., a DNA-targeting sequence) is any polynucleotide sequence having sufficient complementarity with a target polynucleotide sequence to hybridize with the target sequence (e.g., a genomic or mitochondrial DNA target sequence) and direct sequence-specific binding of a complex (e.g., CRISPR complex) to the target sequence. In aspects, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. In aspects, the degree of complementarity between a guide sequence and its corresponding target sequence, when optimally aligned using a suitable alignment algorithm, is at least about 80%, 85%, 90%, 95%, or 100%. In aspects, the degree of complementarity is at least 90%. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies, ELAND (Illumina, San Diego, Calif), SOAP (available at soap.genomics.org.cn), and Maq (available at maq.sourceforge.net). In aspects, a guide sequence is about or more than about 10, 20, 30, 35, 40, 45, 50, 75, or more nucleotides in length. In aspects, a guide sequence is about 10 to about 150, about 15 to about 100 nucleotides in length. In aspects, a guide sequence is less than about 75, 50, 45, 40, 35, 30, 25, 20, 15, 12, or fewer nucleotides in length. In aspects, the guide sequence is about or more than about 20 nucleotides in length. The ability of a guide sequence to direct sequence-specific binding of a complex (e.g., CRISPR complex) to a target sequence may be assessed by any suitable assay. For example, the components of a CRISPR system sufficient to form a complex (e.g., CRISPR complex), including the guide sequence to be tested, may be provided to a host cell having the corresponding target sequence, such as by transfection with vectors encoding the components of the CRISPR sequence, followed by an assessment of preferential cleavage within the target sequence, such as by Surveyor assay known in the art. Similarly, cleavage of a target polynucleotide sequence may be evaluated in a test tube by providing the target sequence, components of a complex (e.g., CRISPR complex), including the guide sequence to be tested and a control guide sequence different from the test guide sequence, and comparing binding or rate of cleavage at the target sequence between the test and control guide sequence reactions. Other assays are possible, and will occur to those skilled in the art. The terms “sgRNA,” “single guide RNA,” and “single guide RNA sequence” are used interchangeably and refer to the polynucleotide sequence including the crRNA sequence and optionally the tracrRNA sequence. The crRNA sequence includes a guide sequence (i.e., “guide” or “spacer”) and a tracr mate sequence (i.e., direct repeat(s)”). The term “guide sequence” refers to the sequence that specifies the target site. In aspects, the two RNA can be encoded separately by a crRNA and tracrRNA as 2 RNA molecules which then form an RNA/RNA complex due to complementary base pairing between the crRNA and tracrRNA (i.e., before being competent to bind to nuclease-deficient RNA-guided DNA endonuclease enzyme). In aspects, a first nucleic acid includes a tracrRNA sequence, and a separate second nucleic acid includes a gRNA sequence lacking a tracrRNA sequence. In aspects, the first nucleic acid including the tracrRNA sequence and the second nucleic acid including the gRNA sequence interact with one another, and optionally are included in a complex (e.g., CRISPR complex).

In general, a tracr mate sequence includes any sequence that has sufficient complementarity with a tracrRNA sequence to promote one or more of: (1) excision of a guide sequence flanked by tracr mate sequences in a cell containing the corresponding tracr sequence; and (2) formation of a complex (e.g., CRISPR complex) at a target sequence, wherein the complex (e.g., CRISPR complex) comprises the tracr mate sequence hybridized to the tracr sequence. In general, degree of complementarity is with reference to the optimal alignment of the tracr mate sequence and tracrRNA sequence, along the length of the shorter of the two sequences. Optimal alignment may be determined by any suitable alignment algorithm, and may further account for secondary structures, such as self-complementarity within either the tracrRNA sequence or tracr mate sequence. In aspects, the degree of complementarity between the tracrRNA sequence and tracr mate sequence along the length of the shorter of the two when optimally aligned is about or more than about 25%, 30%, 40%, 50%, 60%, 70%, 80%, 90%, 95%, 97.5%, 99%, or higher. In aspects, the degree of complementarity is about or at least about 80%, 90%, 95%, or 100%. In aspects, the tracrRNA sequence is about or more than about 5, 10, 15, 20, 30, 40, 50, or more nucleotides in length. In aspects, the tracrRNA sequence and tracr mate sequence are contained within a single transcript, such that hybridization between the two produces a transcript having a secondary structure, such as a hairpin.

The term “RNA-guided DNA endonuclease” and the like refer, in the usual and customary sense, to an enzyme that cleave a phosphodiester bond within a DNA polynucleotide chain, wherein the recognition of the phosphodiester bond is facilitated by a separate RNA sequence (for example, a single guide RNA).

The term “Class II CRISPR endonuclease” refers to endonucleases that have similar endonuclease activity as Cas9 and participate in a Class II CRISPR system. An example Class II CRISPR system is the type II CRISPR locus from Streptococcus pyogenes SF370, which contains a cluster of four genes Cas9, Cas1, Cas2, and Csn1, as well as two non-coding RNA elements, tracrRNA and a characteristic array of repetitive sequences (direct repeats) interspaced by short stretches of non-repetitive sequences (spacers, about 30 bp each). The Cpf1 enzyme belongs to a putative type V CRISPR-Cas system. Both type II and type V systems are included in Class II of the CRISPR-Cas system.

The term “nuclease-deficient RNA-guided DNA endonuclease enzyme” and the like refer, in the usual and customary sense, to an RNA-guided DNA endonuclease (e.g. a mutated form of a naturally occurring RNA-guided DNA endonuclease) that targets a specific phosphodiester bond within a DNA polynucleotide, wherein the recognition of the phosphodiester bond is facilitated by a separate polynucleotide sequence (for example, a RNA sequence (e.g., single guide RNA (sgRNA)), but is incapable of cleaving the target phosphodiester bond to a significant degree (e.g. there is no measurable cleavage of the phosphodiester bond under physiological conditions). A nuclease-deficient RNA-guided DNA endonuclease thus retains DNA-binding ability (e.g. specific binding to a target sequence) when complexed with a polynucleotide (e.g., sgRNA), but lacks significant endonuclease activity (e.g. any amount of detectable endonuclease activity). In aspects, the nuclease-deficient RNA-guided DNA endonuclease enzyme is a CRISPR-associated protein. In aspects, the nuclease-deficient RNA-guided DNA endonuclease enzyme is dCas9, dCas12a, dCpf1, ddCpf1, Cas-phi, a nuclease-deficient Cas9 variant, a nuclease-deficient Class II CRISPR endonuclease, a leucine zipper domain, a winged helix domain, a helix-turn-helix motif, a helix-loop-helix domain, an HMB-box domain, a Wor3 domain, an OB-fold domain, an immunoglobulin domain, or a B3 domain. In aspects, the nuclease-deficient RNA-guided DNA endonuclease enzyme is a leucine zipper domain, a winged helix domain, a helix-turn-helix motif, a helix-loop-helix domain, an HMB-box domain, a Wor3 domain, an OB-fold domain, an immunoglobulin domain, or a B3 domain. In aspects, the nuclease-deficient RNA-guided DNA endonuclease enzyme is a leucine zipper domain. In aspects, the nuclease-deficient RNA-guided DNA endonuclease enzyme is a winged helix domain. In aspects, the nuclease-deficient RNA-guided DNA endonuclease enzyme is a helix-turn-helix motif. In aspects, the nuclease-deficient RNA-guided DNA endonuclease enzyme is a helix-loop-helix domain. In aspects, the nuclease-deficient RNA-guided DNA endonuclease enzyme is an HMB-box domain. In aspects, the nuclease-deficient RNA-guided DNA endonuclease enzyme is a Wor3 domain. In aspects, the nuclease-deficient RNA-guided DNA endonuclease enzyme is an OB-fold domain. In aspects, the nuclease-deficient RNA-guided DNA endonuclease enzyme is an immunoglobulin domain. In aspects, the nuclease-deficient RNA-guided DNA endonuclease enzyme is a B3 domain. In aspects, the nuclease-deficient RNA-guided DNA endonuclease enzyme is dCas9, dCas12a, ddCpf1, Cas-phi, a nuclease-deficient Cas9 variant, or a nuclease-deficient Class II CRISPR endonuclease. In aspects, the nuclease-deficient RNA-guided DNA endonuclease enzyme is dCas9. In aspects, the nuclease-deficient RNA-guided DNA endonuclease enzyme is dCas9 from S. pyogenes. In aspects, the nuclease-deficient RNA-guided DNA endonuclease enzyme is dCas9 from S. aureus. In aspects, the nuclease-deficient RNA-guided DNA endonuclease enzyme is dCas12a. In aspects, the nuclease-deficient RNA-guided DNA endonuclease enzyme is dCas12a from Lachnospiraceae bacterium. In aspects, the nuclease-deficient RNA-guided DNA endonuclease enzyme is dCas12. In aspects, the nuclease-deficient RNA-guided DNA endonuclease enzyme is ddCas12a. In aspects, the nuclease-deficient RNA-guided DNA endonuclease enzyme is Cas-phi.

The term “CRISPR-associated protein” or “CRISPR protein” refers to any CRISPR protein that functions as a nuclease-deficient RNA-guided DNA endonuclease enzyme, i.e., a CRISPR protein in which catalytic sites for endonuclease activity are defective or lack activity. Exemplary CRISPR proteins include dCas9, dCpf1, ddCpf1, dCas12, ddCas12, dCas12a Cas-phi, a nuclease-deficient Cas9 variant, a nuclease-deficient Class II CRISPR endonuclease, and the like.

The term “nuclease-deficient DNA endonuclease enzyme” refers to a DNA endonuclease (e.g. a mutated form of a naturally occurring DNA endonuclease) that targets a specific phosphodiester bond within a DNA polynucleotide, but that does not require an RNA guide. In embodiments, the “nuclease-deficient DNA endonuclease enzyme” is a zinc finger domain or a transcription activator-like effector (TALE).

In embodiments, the nuclease-deficient DNA endonuclease enzyme is a “zinc finger domain.” The term “zinc finger domain” or “zinc finger binding domain” or “zinc finger DNA binding domain” are used interchangeably and refer to a protein, or a domain within a larger protein, that binds DNA in a sequence-specific manner through one or more zinc fingers, which are regions of amino acid sequence within the binding domain whose structure is stabilized through coordination of a zinc ion. In embodiments, the zinc finger domain is non-naturally occurring in that it is engineered to bind to a target site of choice. In aspects, the zinc finger binding domain refers to a protein, a domain within a larger protein, or a nuclease-deficient RNA-guided DNA endonuclease enzyme that is capable of binding to any zinc finger known in the art, such as the C2H2 type, the CCHC type, the PHD type, or the RING type of zinc fingers.

As used herein, a “zinc finger” is a polypeptide structural motif folded around a bound zinc cation. In embodiments, the polypeptide of a zinc finger has a sequence of the form X₃-Cys-X₂₋₄-Cys-X₁₂-His-X₃₋₅-His-X₄, wherein X is any amino acid (e.g., X₂-4 indicates an oligopeptide 2-4 amino acids in length). There is generally a wide range of sequence variation in the 28-31 amino acids of the known zinc finger polypeptides. Only the two consensus histidine residues and two consensus cysteine residues bound to the central zinc atom are invariant. Of the remaining residues, three to five are highly conserved, while there may be significant variation among the other residues. Despite the wide range of sequence variation in the polypeptide, zinc fingers of this type have a similar three dimensional structure. However, there is a wide range of binding specificities among the different zinc fingers, i.e. different zinc fingers bind double stranded polynucleotides having a wide range of nucleotides sequences. In aspects, the zinc finger is the C2H2 type. In aspects, the zinc finger is the CCHC type. In aspects, the zinc finger is the PHD type. In aspects, the zinc finger is the RING type.

In embodiments, the nuclease-deficient DNA endonuclease enzyme is a TALE. “TALE” or “transcription activator-like effector” refer to artificial restriction enzymes generated by fusing the TAL effector DNA binding domain to a DNA cleavage domain. TALEs enable efficient, programmable, and specific DNA cleavage and represent powerful tools for genome editing in situ. Transcription activator-like effectors (TALEs) can be quickly engineered to bind practically any DNA. sequence. The term TALE, as used herein, is broad and includes a monomeric TALE that can cleave double stranded DNA without assistance from another TALE. The term TALE is also used to refer to one or both members of a pair of TALEs that are engineered to work together to cleave DNA at the same site. TALEs that work together may be referred to as a left-TALE and a right-TALE, which references the handedness of DNA. TALE are proteins secreted by Xanthomonas bacteria. The DNA binding domain contains a highly conserved 33-34 amino acid sequence with the exception of the 12th and 13th amino acids. These two locations are highly variable (repeat variable diresidue (MUD)) and show a strong correlation with specific nucleotide recognition. This simple relationship between amino acid sequence and DNA recognition has allowed for the engineering of specific DNA binding domains by selecting a combination of repeat segments containing the appropriate RVDs.

In embodiments, the nuclease-deficient RNA-guided DNA endonuclease enzyme is dCas9. The terms “dCas9” or “dCas9 protein” as referred to herein is a Cas9 protein in which both catalytic sites for endonuclease activity are defective or lack activity. In aspects, the dCas9 protein has mutations at positions corresponding to D10A and H840A of S. pyogenes Cas9. In aspects, the dCas9 protein lacks endonuclease activity due to point mutations at both endonuclease catalytic sites (RuvC and HNH) of wild type Cas9. The point mutations can be D10A and H840A. In aspects, the dCas9 has substantially no detectable endonuclease (e.g., endodeoxyribonuclease) activity.

A “CRISPR associated protein 9,” “Cas9,” “Csn1” or “Cas9 protein” as referred to herein includes any of the recombinant or naturally-occurring forms of the Cas9 endonuclease or variants or homologs thereof that maintain Cas9 endonuclease enzyme activity (e.g. within at least 50%, 80%, 90%, 95%, 96%, 97%, 98%, 99% or 100% activity compared to Cas9). In aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Cas9 protein. In aspects, the Cas9 protein is substantially identical to the protein identified by the UniProt reference number Q99ZW2 or a variant or homolog having substantial identity thereto. In aspects, the Cas9 protein has at least 75% sequence identity to the amino acid sequence of the protein identified by the UniProt reference number Q99ZW2. In aspects, the Cas9 protein has at least 80% sequence identity to the amino acid sequence of the protein identified by the UniProt reference number Q99ZW2. In aspects, the Cas9 protein has at least 85% sequence identity to the amino acid sequence of the protein identified by the UniProt reference number Q99ZW2. In aspects, the Cas9 protein has at least 90% sequence identity to the amino acid sequence of the protein identified by the UniProt reference number Q99ZW2. In aspects, the Cas9 protein has at least 95% sequence identity to the amino acid sequence of the protein identified by the UniProt reference number Q99ZW2.

In embodiments, the nuclease-deficient RNA-guided DNA endonuclease enzyme is “ddCpf1” or “ddCas12a”. The terms “DNAse-dead Cpf1” or “ddCpf1” refer to mutated Acidaminococcus sp. Cpf1 (AsCpf1) resulting in the inactivation of Cpf1 DNAse activity. In aspects, ddCpf1 includes an E993A mutation in the RuvC domain of AsCpf1. In aspects, the ddCpf1 has substantially no detectable endonuclease (e.g., endodeoxyribonuclease) activity.

In embodiments, the nuclease-deficient RNA-guided DNA endonuclease enzyme is dLbCpf1. The term “dLbCpf1: refers to mutated Cpf1 from Lachnospiraceae bacterium ND2006 (LbCpf1) that lacks DNAse activity. In aspects, dLbCpf1 includes a D832A mutation. In aspects, the dLbCpf1 has substantially no detectable endonuclease (e.g., endodeoxyribo-nuclease) activity.

In embodiments, the nuclease-deficient RNA-guided DNA endonuclease enzyme is dFnCpf1. The term “dFnCpf1” refers to mutated Cpf1 from Francisella novicida U112 (FnCpf1) that lacks DNAse activity. In aspects, dFnCpf1 includes a D917A mutation. In aspects, the dFnCpf1 has substantially no detectable endonuclease (e.g., endodeoxyribo-nuclease) activity.

A “Cpf1” or “Cpf1 protein” as referred to herein includes any of the recombinant or naturally-occurring forms of the Cpf1 (CRISPR from Prevotella and Francisella 1) endonuclease or variants or homologs thereof that maintain Cpf1 endonuclease enzyme activity (e.g. within at least 50%, 80%, 90%, 95%, 96%, 97%, 98%, 99% or 100% activity compared to Cpf1). In aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Cpf1 protein. In aspects, the Cpf1 protein is substantially identical to the protein identified by the UniProt reference number U2UMQ6 or a variant or homolog having substantial identity thereto. In aspects, the Cpf1 protein is identical to the protein identified by the UniProt reference number U2UMQ6. In aspects, the Cpf1 protein has at least 75% sequence identity to the amino acid sequence of the protein identified by the UniProt reference number U2UMQ6. In aspects, the Cpf1 protein has at least 80% sequence identity to the amino acid sequence of the protein identified by the UniProt reference number U2UMQ6. In aspects, the Cpf1 protein is identical to the protein identified by the UniProt reference number U2UMQ6. In aspects, the Cpf1 protein has at least 85% sequence identity to the amino acid sequence of the protein identified by the UniProt reference number U2UMQ6. In aspects, the Cpf1 protein is identical to the protein identified by the UniProt reference number U2UMQ6. In aspects, the Cpf1 protein has at least 90% sequence identity to the amino acid sequence of the protein identified by the UniProt reference number U2UMQ6. In aspects, the Cpf1 protein is identical to the protein identified by the UniProt reference number U2UMQ6. In aspects, the Cpf1 protein has at least 95% sequence identity to the amino acid sequence of the protein identified by the UniProt reference number U2UMQ6.

In embodiments, the nuclease-deficient RNA-guided DNA endonuclease enzyme is a nuclease-deficient Cas9 variant. The term “nuclease-deficient Cas9 variant” refers to a Cas9 protein having one or more mutations that increase its binding specificity to PAM compared to wild type Cas9 and further include mutations that render the protein incapable of or having severely impaired endonuclease activity. Without wishing to be bound by theory, it is believed that the target sequence should be associated with a PAM (protospacer adjacent motif); that is, a short sequence recognized by the CRISPR complex. The precise sequence and length requirements for the PAM differ depending on the CRISPR enzyme used, but PAMs are typically 2-5 base pair sequences adjacent the protospacer (that is, the target sequence). The binding specificity of nuclease-deficient Cas9 variants to PAM can be determined by any method known in the art. Descriptions and uses of known Cas9 variants may be found, for example, in Shmakov et al., Diversity and evolution of class 2 CRISPR-Cas systems. Nat. Rev. Microbiol. 15, 2017 and Cebrian-Serrano et al, CRISPR-Cas orthologues and variants: optimizing the repertoire, specificity and delivery of genome engineering tools. Mamm. Genome 7-8, 2017, which are incorporated herein by reference in their entirety and for all purposes.

In embodiments, the nuclease-deficient RNA-guided DNA endonuclease enzyme is a nuclease-deficient Class II CRISPR endonuclease. The term “nuclease-deficient Class II CRISPR endonuclease” as used herein refers to any Class II CRISPR endonuclease having mutations resulting in reduced, impaired, or inactive endonuclease activity.

The term “antibody” is used according to its commonly known meaning in the art. Antibodies exist, e.g., as intact immunoglobulins or as a number of well-characterized fragments produced by digestion with various peptidases. Thus, for example, pepsin digests an antibody below the disulfide linkages in the hinge region to produce F(ab)′2, a dimer of Fab which itself is a light chain joined to VH-CH1 by a disulfide bond. The F(ab)′2 may be reduced under mild conditions to break the disulfide linkage in the hinge region, thereby converting the F(ab)′2 dimer into an Fab′ monomer. The Fab′ monomer is essentially Fab with part of the hinge region. While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries.

Antibodies are large, complex molecules (molecular weight of −150,000 or about 1320 amino acids) with intricate internal structure. A natural antibody molecule contains two identical pairs of polypeptide chains, each pair having one light chain and one heavy chain. Each light chain and heavy chain in turn consists of two regions: a variable (“V”) region involved in binding the target antigen, and a constant (“C”) region that interacts with other components of the immune system. The light and heavy chain variable regions come together in 3-dimensional space to form a variable region that binds the antigen (for example, a receptor on the surface of a cell). Within each light or heavy chain variable region, there are three short segments (averaging 10 amino acids in length) called the complementarity determining regions (“CDRs”). The six CDRs in an antibody variable domain (three from the light chain and three from the heavy chain) fold up together in 3-dimensional space to form the actual antibody binding site which docks onto the target antigen. The position and length of the CDRs have been precisely defined by Kabat, E. et al., Sequences of Proteins of Immunological Interest, U.S. Department of Health and Human Services, 1983, 1987.

An “antibody variant” as provided herein refers to a polypeptide capable of binding to an antigen and including one or more structural domains (e.g., light chain variable domain, heavy chain variable domain) of an antibody or fragment thereof. Non-limiting examples of antibody variants include single-domain antibodies or nanobodies, monospecific Fab2, bispecific Fab2, trispecific Fab3, monovalent IgGs, scFv, bispecific antibodies, bispecific diabodies, trispecific triabodies, scFv-Fc, minibodies, IgNAR, V-NAR, hcIgG, VhH, or peptibodies. A “peptibody” as provided herein refers to a peptide moiety attached (through a covalent or non-covalent linker) to the Fc domain of an antibody.

Antibodies exist, for example, as intact immunoglobulins or as a number of well-characterized fragments produced by digestion with various peptidases. Thus, for example, pepsin digests an antibody below the disulfide linkages in the hinge region to produce F(ab)′2, a dimer of Fab which itself is a light chain joined to VH-CH1 by a disulfide bond. The F(ab)′2 may be reduced under mild conditions to break the disulfide linkage in the hinge region, thereby converting the F(ab)′2 dimer into an Fab′ monomer. The Fab′ monomer is essentially the antigen binding portion with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

A single-chain variable fragment (scFv) is typically a fusion protein of the variable regions of the heavy (VH) and light chains (VL) of immunoglobulins, connected with a short linker peptide of 10 to about 25 amino acids. The linker may usually be rich in glycine for flexibility, as well as serine or threonine for solubility. The linker can either connect the N-terminus of the VH with the C-terminus of the VL, or vice versa.

The epitope of an antibody is the region of its antigen to which the antibody binds. Two antibodies bind to the same or overlapping epitope if each competitively inhibits (blocks) binding of the other to the antigen. That is, a 1×, 5×, 10×, 20× or 100× excess of one antibody inhibits binding of the other by at least 30% but preferably 50%, 75%, 90% or even 99% as measured in a competitive binding assay (see, e.g., Junghans et al., Cancer Res. 50:1495, 1990). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

A “control” sample or value refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). A control can also represent an average value gathered from a number of tests or results. One of skill in the art will recognize that controls can be designed for assessment of any number of parameters. For example, a control can be devised to compare therapeutic benefit based on pharmacological data (e.g., half-life) or therapeutic measures (e.g., comparison of side effects). Controls are also valuable for determining the significance of data. For example, if values for a given parameter are widely variant in controls, variation in test samples will not be considered as significant.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms; fully or partially remove the disease's underlying cause; shorten a disease's duration; or do a combination of these things.

“Treating” and “treatment” as used herein also include prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient. In embodiments, the treating or treatment is not prophylactic treatment.

“Patient” or “subject in need thereof” refers to a living organism suffering from or prone to a disease or condition that can be treated by administration of a pharmaceutical composition as provided herein. Non-limiting examples include humans, other mammals, bovines, rats, mice, dogs, cats, and other non-mammalian animals. In embodiments, a patient is human.

An “effective amount” is an amount sufficient for a compound to accomplish a stated purpose relative to the absence of the compound (e.g., achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

The term “therapeutically effective amount,” as used herein, refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

Dosages may be varied depending upon the requirements of the patient and the compound being employed. The dose administered to a patient, in the context of the present disclosure, should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

The term “administering” is used in accordance with its plain and ordinary meaning and includes oral administration, administration by inhalation, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In embodiments, the administering does not include administration of any active agent other than the recited active agent.

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated; however, the resulting reaction product can be produced directly from a reaction between the added reagents or from an intermediate from one or more of the added reagents that can be produced in the reaction mixture.

The term “inhibition”, “inhibit”, “inhibiting” and the like in reference to a protein-inhibitor interaction means negatively affecting (e.g., decreasing) the activity or function of the protein relative to the activity or function of the protein in the absence of the inhibitor. In embodiments inhibition means negatively affecting (e.g., decreasing) the concentration or levels of the protein relative to the concentration or level of the protein in the absence of the inhibitor. In embodiments inhibition refers to reduction of a disease or symptoms of disease. In embodiments, inhibition refers to a reduction in the activity of a particular protein target. Thus, inhibition includes, at least in part, partially or totally blocking stimulation, decreasing, preventing, or delaying activation, or inactivating, desensitizing, or down-regulating signal transduction or enzymatic activity or the amount of a protein. In embodiments, inhibition refers to a reduction of activity of a target protein resulting from a direct interaction (e.g. an inhibitor binds to the target protein). In embodiments, inhibition refers to a reduction of activity of a target protein from an indirect interaction (e.g. an inhibitor binds to a protein that activates the target protein, thereby preventing target protein activation). In embodiments, the KIAA0930 inhibitor described herein inhibits expression of a KIAA0930 protein. In embodiments, the KIAA0930 inhibitor described herein reduces expression of a KIAA0930 protein. In embodiments, the KIAA0930 inhibitor described herein suppresses expression of a KIAA0930 protein.

The term “associated” or “associated with” in the context of a substance or substance activity or function associated with a disease (e.g., a protein associated with an infectious disease) means that the disease (e.g., cancer, inflammatory disease, autoimmune disease, or infectious disease) is caused by (in whole or in part), or a symptom of the disease is caused by (in whole or in part) the substance or substance activity or function. As used herein, what is described as being associated with a disease, if a causative agent, could be a target for treatment of the disease.

The term “signaling pathway” as used herein refers to a series of interactions between cellular and optionally extra-cellular components (e.g. proteins, nucleic acids, small molecules, ions, lipids) that conveys a change in one component to one or more other components, which in turn may convey a change to additional components, which is optionally propagated to other signaling pathway components.

The term “exogenous” refers to a molecule or substance (e.g., a compound, nucleic acid or protein) that originates from outside a given cell or organism. For example, an “exogenous promoter” as referred to herein is a promoter that does not originate from the plant it is expressed by. Conversely, the term “endogenous” or “endogenous promoter” refers to a molecule or substance that is native to, or originates within, a given cell or organism.

“Pharmaceutically acceptable excipient” and “pharmaceutically acceptable carrier” refer to a substance that aids the administration of an active agent to and absorption by a subject and can be included in the compositions described herein without causing a significant adverse toxicological effects on the subject. In embodiments, the pharmaceutically acceptable excipient include one or more pharmaceutically acceptable additives. Non-limiting examples of pharmaceutically acceptable excipients include water, NaC1, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylase or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compositions described herein. One of skill in the art will recognize that additional pharmaceutical excipients may be useful. The term “pharmaceutically acceptable salt” refers to salts derived from a variety of organic and inorganic counter ions well known in the art and include, by way of example only, sodium, potassium, calcium, magnesium, ammonium, tetraalkylammonium, and the like.

The terms “bind” and “bound” as used herein is used in accordance with its plain and ordinary meaning and refers to the association between atoms or molecules. The association can be direct or indirect. For example, bound atoms or molecules may be bound, e.g., by covalent bond, linker (e.g. a first linker or second linker), or non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like).

The term “capable of binding” as used herein refers to a moiety (e.g. a compound as described herein) that is able to measurably bind to a target (e.g., a NF-κB, a Toll-like receptor protein). In embodiments, where a moiety is capable of binding a target, the moiety is capable of binding with a Kd of less than about 10 μM, 5 μM, 1 μM, 500 nM, 250 nM, 100 nM, 75 nM, 50 nM, 25 nM, 15 nM, 10 nM, 5 nM, 1 nM, or about 0.1 nM.

The term “conjugated” when referring to two moieties means the two moieties are bonded, wherein the bond or bonds connecting the two moieties may be covalent or non-covalent. In embodiments, the two moieties are covalently bonded to each other (e.g. directly or through a covalently bonded intermediary). In embodiments, the two moieties are non-covalently bonded (e.g. through ionic bond(s), Van Der Waal's bond(s)/interactions, hydrogen bond(s), polar bond(s), or combinations or mixtures thereof).

The term “heterologous” when used with reference to portions of a nucleic acid indicates that the nucleic acid including two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein including two or more subsequences that are not found in the same relationship to each other in nature (e.g., a fusion protein).

The phrase “specifically (or selectively) binds” to an antibody or “specifically (or selectively) immunoreactive with,” when referring to a protein or peptide refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

The terms “isolate” or “isolated”, when applied to a nucleic acid, virus, or protein, denotes that the nucleic acid, virus, or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. An RNA that is the predominant species present in a preparation is substantially purified.

A “detectable agent” or “detectable moiety” is a compound or composition detectable by appropriate means such as spectroscopic, photochemical, biochemical, immunochemical, chemical, magnetic resonance imaging, or other physical means. The RNA described herein and the expression level of the RNA described herein may be accomplished through the use of a detectable moiety in an assay or kit. A detectable moiety is a monovalent detectable agent or a detectable agent bound (e.g. covalently and directly or via a linking group) with another compound, e.g., a nucleic acid. Exemplary detectable agents/moieties for use in the present disclosure include an antibody ligand, a peptide, a nucleic acid, radioisotopes, paramagnetic metal ions, fluorophore (e.g. fluorescent dyes), electron-dense reagents, enzymes (e.g., as commonly used in an ELISA), biotin, a biotin-avidin complex, a biotin-streptavidin complex, digoxigenin, magnetic beads (e.g., DYNABEADS® by ThermoFisher, encompassing functionalized magnetic beads such as DYNABEADS® M-270 amine by ThermoFisher), paramagnetic molecules, paramagnetic nanoparticles, ultrasmall superparamagnetic iron oxide nanoparticles, ultrasmall superparamagnetic iron oxide nanoparticle aggregates, superparamagnetic iron oxide nanoparticles, superparamagnetic iron oxide nanoparticle aggregates, monocrystalline iron oxide nanoparticles, monocrystalline iron oxide, nanoparticle contrast agents, liposomes or other delivery vehicles containing Gadolinium chelate molecules, gadolinium, radionuclides (e.g. carbon-11, nitrogen-13, oxygen-15, fluorine-18, rubidium-82), fluorodeoxyglucose (e.g. fluorine-18 labeled), any gamma ray emitting radionuclides, positron-emitting radionuclide, radiolabeled glucose, radiolabeled water, radiolabeled ammonia, biocolloids, microbubbles (e.g. including microbubble shells including albumin, galactose, lipid, and/or polymers; microbubble gas core including air, heavy gas(es), perfluorcarbon, nitrogen, octafluoropropane, perflexane lipid microsphere, perflutren, etc.), iodinated contrast agents (e.g. iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide.

“Biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. In embodiments, a biological sample is blood. In embodiments, a biological sample is a serum sample (e.g., the fluid and solute component of blood without the clotting factors). In embodiments, a biological sample is a plasma sample (e.g, the liquid portion of blood).

The term “prevent” is used in accordance with its plain and ordinary meaning and refers to a decrease in the occurrence of disease symptoms in a patient. The prevention may be complete (no detectable symptoms) or partial, such that fewer symptoms are observed than would likely occur absent treatment.

The term “disease” or “condition” refers to a state of being or health status of a patient or subject that is being treated with the compounds or methods provided herein. The disease may be a cancer. The disease may be an autoimmune disease. The disease may be an inflammatory disease. The disease may be an infectious disease. In instances, “cancer” refers to human cancers and carcinomas, sarcomas, adenocarcinomas, lymphomas, leukemias, etc., including solid and lymphoid cancers, kidney, breast, lung, bladder, colon, ovarian, prostate, pancreas, stomach, brain, head and neck, skin, uterine, testicular, glioma, esophagus, and liver cancer, including hepatocarcinoma, lymphoma, including B-acute lymphoblastic lymphoma, non-Hodgkin's lymphomas (e.g., Burkitt's, Small Cell, and Large Cell lymphomas), Hodgkin's lymphoma, leukemia (including AML, ALL, and CML), or multiple myeloma.

The term “cancer” is used in accordance with its plain ordinary meaning and refers to all types of cancer, neoplasm or malignant tumors found in mammals (e.g. humans), including leukemias, lymphomas, carcinomas and sarcomas. Exemplary cancers that may be treated with a compound or method provided herein include brain cancer, glioma, glioblastoma, neuroblastoma, prostate cancer, colorectal cancer, pancreatic cancer, Medulloblastoma, melanoma, cervical cancer, gastric cancer, ovarian cancer, lung cancer, cancer of the head, Hodgkin's Disease, and Non-Hodgkin's Lymphomas. Exemplary cancers that may be treated with a compound or method provided herein include cancer of the thyroid, endocrine system, brain, breast, cervix, colon, head & neck, liver, kidney, lung, ovary, pancreas, rectum, stomach, and uterus. Additional examples include, thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, non-small cell lung carcinoma, mesothelioma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

The term “inflammatory cytokine” as used herein refers to cytokines produced by T helper cells (T h) and macrophages and involved in the upregulation of inflammatory reactions. Cytokines are proteins made in response to pathogens and other antigens that regulate and mediate inflammatory and immune responses. Proinflammatory cytokines include, but are not limited to, interleukin-1 (IL-1), IL-6, IL-12, IL-18, tumor necrosis factor alpha (TNF-α), interferon gamma (IFNγ), and granulocyte-macrophage colony stimulating factor (GM-CSF) and play an important role in mediating the innate immune response. Inflammatory cytokines are predominantly produced by and involved in the upregulation of inflammatory reactions. Excessive chronic production of inflammatory cytokines contribute to inflammatory diseases, including arthritis, autoimmunity, atherosclerosis and cancer. Dysregulation has also been linked to depression and other neurological diseases. A balance between proinflammatory and anti-inflammatory cytokines is necessary to maintain health. Aging and exercise also play a role in the amount of inflammation from the release of proinflammatory cytokines.

The term “chemotherapy” refers to a type of cancer treatment that uses one or more anti-cancer drugs (chemotherapeutic agents) as part of a standardized chemotherapy regimen. Chemotherapy may be given with a curative intent (which almost always involves combinations of drugs), or it may aim to prolong life or to reduce symptoms (palliative chemotherapy). Chemotherapy drugs include, but are not limited to, alkylating agents, nitrosoureas, antimetabolites, alkaloids, antitumor antibiotics, hormonal agents and biological response modifiers. Examples of chemotherapy drugs include, but are not limited to, cyclophosphamide, melphalan, temozolomide, carboplatin, cisplatin, oxaliplatin, 5-fluorouracil, 6-mercaptopurine, cytarabine, gemcitabine, methotrexate, actimycin-D, blemycin, daunorubicin, doxorubicin, docetaxel, estramustine, paclitaxel, vinblastine, etoposide, irinotecan, teniposide, topotecan, prednisone, methylprednisolone and dexamethasone. Traditional chemotherapeutic agents are cytotoxic by means of interfering with cell division (mitosis) but cancer cells vary widely in their susceptibility to these agents. Many of the side effects of chemotherapy can be traced to damage to normal cells that divide rapidly and are thus sensitive to anti-mitotic drugs such as, but not limited to, cells in the bone marrow, digestive tract and hair follicles. In embodiments, the chemotherapy includes administration of an effective amount of an anticancer agent as set forth herein.

“Anti-cancer agent” and “anticancer agent” are used in accordance with their plain ordinary meaning and refers to a composition (e.g. compound, drug, antagonist, inhibitor, modulator) having antineoplastic properties or the ability to inhibit the growth or proliferation of cells. In some embodiments, an anti-cancer agent is a chemotherapeutic. In some embodiments, an anti-cancer agent is an agent identified herein having utility in methods of treating cancer. In some embodiments, an anti-cancer agent is an agent approved by the FDA or similar regulatory agency of a country other than the USA, for treating cancer. Examples of anti-cancer agents include, but are not limited to, MEK (e.g. MEK1, MEK2, or MEK1 and MEK2) inhibitors (e.g. XL518, CI-1040, PD035901, selumetinib/AZD6244, GSK1120212/trametinib, GDC-0973, ARRY-162, ARRY-300, AZD8330, PD0325901, U0126, PD98059, TAK-733, PD318088, AS703026, BAY 869766), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, meiphalan), ethylenimine and methylmelamines (e.g., hexamethlymelamine, thiotepa), alkyl sulfonates (e.g., busulfan), nitrosoureas (e.g., carmustine, lomusitne, semustine, streptozocin), triazenes (decarbazine)), anti-metabolites (e.g., 5-azathioprine, leucovorin, capecitabine, fludarabine, gemcitabine, pemetrexed, raltitrexed, folic acid analog (e.g., methotrexate), or pyrimidine analogs (e.g., fluorouracil, floxouridine, Cytarabine), purine analogs (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds (e.g. cisplatin, oxaloplatin, carboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide), epipodophyllotoxins (e.g., etoposide), antibiotics (e.g., daunorubicin, doxorubicin, bleomycin), enzymes (e.g., L-asparaginase), inhibitors of mitogen-activated protein kinase signaling (e.g. U0126, PD98059, PD184352, PD0325901, ARRY-142886, SB239063, SP600125, BAY 43-9006, wortmannin, or LY294002, Syk inhibitors, mTOR inhibitors, antibodies (e.g., rituxan), gossyphol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec®), geldanamycin, 17-N-Allylamino-17-Demethoxygeldanamycin (17-AAG), flavopiridol, LY294002, bortezomib, trastuzumab, BAY 11-7082, PKC412, PD184352, 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B 1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer, Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; iimofosine; interleukin (including recombinant interleukin II, or r1L.sub.2), interferon alfa-2a; interferon alfa-2b; interferon alfa-n1; interferon alfa-n3; interferon beta-1a; interferon gamma-1b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazoie; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride, agents that arrest cells in the G2-M phases and/or modulate the formation or stability of microtubules, (e.g. Taxol™ (i.e. paclitaxel), Taxotere™, compounds comprising the taxane skeleton, Erbulozole (i.e. R-55104), Dolastatin 10 (i.e. DLS-10 and NSC-376128), Mivobulin isethionate (i.e. as CI-980), Vincristine, NSC-639829, Discodermolide (i.e. as NVP-XX-A-296), ABT-751 (Abbott, i.e. E-7010), Altorhyrtins (e.g. Altorhyrtin A and Altorhyrtin C), Spongistatins (e.g. Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (i.e. LU-103793 and NSC-D-669356), Epothilones (e.g. Epothilone A, Epothilone B, Epothilone C (i.e. desoxyepothilone A or dEpoA), Epothilone D (i.e. KOS-862, dEpoB, and desoxyepothilone B), Epothilone E, Epothilone F, Epothilone B N-oxide, Epothilone A N-oxide, 16-aza-epothilone B, 21-aminoepothilone B (i.e. BMS-310705), 21-hydroxyepothilone D (i.e. Desoxyepothilone F and dEpoF), 26-fluoroepothilone, Auristatin PE (i.e. NSC-654663), Soblidotin (i.e. TZT-1027), LS-4559-P (Pharmacia, i.e. LS-4577), LS-4578 (Pharmacia, i.e. LS-477-P), LS-4477 (Pharmacia), LS-4559 (Pharmacia), RPR-112378 (Aventis), Vincristine sulfate, DZ-3358 (Daiichi), FR-182877 (Fujisawa, i.e. WS-9885B), GS-164 (Takeda), GS-198 (Takeda), KAR-2 (Hungarian Academy of Sciences), BSF-223651 (BASF, i.e. ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (i.e. LY-355703), AC-7739 (Ajinomoto, i.e. AVE-8063A and CS-39.HCl), AC-7700 (Ajinomoto, i.e. AVE-8062, AVE-8062A, CS-39-L-Ser.HCl, and RPR-258062A), Vitilevuamide, Tubulysin A, Canadensol, Centaureidin (i.e. NSC-106969), T-138067 (Tularik, i.e. T-67, TL-138067 and TI-138067), COBRA-1 (Parker Hughes Institute, i.e. DDE-261 and WHI-261), H10 (Kansas State University), H16 (Kansas State University), Oncocidin A1 (i.e. BTO-956 and DIME), DDE-313 (Parker Hughes Institute), Fijianolide B, Laulimalide, SPA-2 (Parker Hughes Institute), SPA-1 (Parker Hughes Institute, i.e. SPIKET-P), 3-IAABU (Cytoskeleton/Mt. Sinai School of Medicine, i.e. MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, i.e. MF-191), TMPN (Arizona State University), Vanadocene acetylacetonate, T-138026 (Tularik), Monsatrol, lnanocine (i.e. NSC-698666), 3-IAABE (Cytoskeleton/Mt. Sinai School of Medicine), A-204197 (Abbott), T-607 (Tuiarik, i.e. T-900607), RPR-115781 (Aventis), Eleutherobins (such as Desmethyleleutherobin, Desaetyleleutherobin, lsoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (i.e. NSCL-96F037), D-68838 (Asta Medica), D-68836 (Asta Medica), Myoseverin B, D-43411 (Zentaris, i.e. D-81862), A-289099 (Abbott), A-318315 (Abbott), HTI-286 (i.e. SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-250411 (Sanofi)), steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Guérin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), immunotherapy (e.g., cellular immunotherapy, antibody therapy, cytokine therapy, combination immunotherapy, etc.), radioimmunotherapy (e.g., anti-CD20 monoclonal antibody conjugated to 111In, 90Y, or 131I, etc.), immune checkpoint inhibitors (e.g., CTLA4 blockade, PD-1 inhibitors, PD-L1 inhibitors, etc.), triptolide, homoharringtonine, dactinomycin, doxorubicin, epirubicin, topotecan, itraconazole, vindesine, cerivastatin, vincristine, deoxyadenosine, sertraline, pitavastatin, irinotecan, clofazimine, 5-nonyloxytryptamine, vemurafenib, dabrafenib, erlotinib, gefitinib, EGFR inhibitors, epidermal growth factor receptor (EGFR)-targeted therapy or therapeutic (e.g. gefitinib (Iressa™), erlotinib (Tarceva™), cetuximab (Erbitux™), lapatinib (Tykerb™), panitumumab (Vectibix™), vandetanib (Caprelsa™) afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626), sorafenib, imatinib, sunitinib, dasatinib, or the like.

The terms an “elevated level” or an “increased level” or a “high level” of gene expression is an expression level of the gene or protein that is higher than the expression level of the gene or protein in a standard control or in a control with no or very low risk of recurrence (e.g. a control biological sample derived from a subject or subjects with no or low risk of recurrence). The standard control may be any suitable control, examples of which are described herein. The control with no risk of recurrence may be a patient or subject who has undergone surgery for treatment of cancer and is at no risk or very low risk of developing cancer recurrence within the first 5 years after surgery, examples of which are described herein.

The terms a “reduced level” or a “decreased expression level” or a “low level” of gene expression is an expression level of the gene or protein that is lower than the expression level of the gene or protein in a standard control or in a control with no risk of recurrence. The standard control may be any suitable control, examples of which are described herein. The control with no risk of recurrence is a patient or subject who has undergone surgery for treatment of cancer and is at no risk or very low risk of developing cancer recurrence within the first 5 years after surgery, examples of which are described herein.

“Pathway” refers to a set of system components involved in two or more sequential molecular interactions that result in the production of a product or activity. A pathway can produce a variety of products or activities that can include, for example, intermolecular interactions, changes in expression of a nucleic acid or polypeptide, the formation or dissociation of a complex between two or more molecules, accumulation or destruction of a metabolic product, activation or deactivation of an enzyme or binding activity. Thus, the term “pathway” includes a variety of pathway types, such as, for example, a biochemical pathway, a gene expression pathway, and a regulatory pathway. Similarly, a pathway can include a combination of these exemplary pathway types.

The term “tissue sample” is used in accordance with its plain ordinary meaning and refers to a piece of tissue removed from an organism for examination, analysis, or propagation.

“Assaying” or “detecting” means using an analytical procedure to qualitatively assess or quantitatively measure the level of a gene or a protein as described herein such as, for example, detecting a KIAA0930 protein or a KIAA0930 gene, using an analytical procedure (such as an in vitro procedure) to qualitatively assess or quantitatively measure the level of the selected protein or gene. In embodiments, the detecting includes or is assaying, which includes wet lab analysis, physical steps and/or physical manipulation of the sample, for example in a laboratory setting involving physical assaying techniques.

Prevention or Treatment of Involuntary Depletion of Adipose Tissue and Muscle Mass

Provided herein, inter alia, are methods which prevent or treat a wasting syndrome in a subject in need thereof. These methods are effective in preventing and treating involuntary depletion of adipose tissue and muscle mass. The disclosed methods comprise administering to the subject an effective amount of a KIAA0930 inhibitor.

In embodiments, the KIAA0930 inhibitor suppresses or reduces expression of the KIAA0930 protein or the KIAA0930 mRNA.

In embodiments, the KIAA0930 inhibitor is a short-hairpin RNA (shRNA), a small interference RNA (siRNA), a piwi-interacting RNA (piRNA), a microRNA (miRNA), an antisense oligonucleotide, such as a GapmeR or a morpholinooligonucleotide, a CRISPR Cas guide RNA (gRNA), or a small molecule compound.

In embodiments, the KIAA0930 inhibitor is a shRNA. In embodiments, the shRNA comprises SEQ ID NO:8. In embodiments, the shRNA comprises a sequence that is at least 80% identical to SEQ ID NO:8. In embodiments, the shRNA comprises a sequence that is at least 85% identical to SEQ ID NO:8. In embodiments, the shRNA comprises a sequence that is at least 90% identical to SEQ ID NO:8. In embodiments, the shRNA comprises a sequence that is at least 95% identical to SEQ ID NO:8. In embodiments, the shRNA comprises a sequence that is at least 98% identical to SEQ ID NO:8. In embodiments, the shRNA comprises a sequence that is 100% identical to SEQ ID NO:8. In embodiments, the shRNA comprises SEQ ID NO:9. In embodiments, the shRNA comprises a sequence that is at least 80% identical to SEQ ID NO:9. In embodiments, the shRNA comprises a sequence that is at least 85% identical to SEQ ID NO:9. In embodiments, the shRNA comprises a sequence that is at least 90% identical to SEQ ID NO:9. In embodiments, the shRNA comprises a sequence that is at least 95% identical to SEQ ID NO:9. In embodiments, the shRNA comprises a sequence that is at least 98% identical to SEQ ID NO:9. In embodiments, the shRNA comprises a sequence that is 100% identical to SEQ ID NO:9.

In embodiments, the KIAA0930 inhibitor is a small interference RNA (siRNA). In embodiments, the siRNA comprises SEQ ID NO:1. In embodiments, the siRNA comprises a sequence that is at least 80% identical to SEQ ID NO:1. In embodiments, the siRNA comprises a sequence that is at least 85% identical to SEQ ID NO:1. In embodiments, the siRNA comprises a sequence that is at least 90% identical to SEQ ID NO:1. In embodiments, the siRNA comprises a sequence that is at least 95% identical to SEQ ID NO:1. In embodiments, the siRNA comprises a sequence that is at least 98% identical to SEQ ID NO:1. In embodiments, the siRNA comprises a sequence that is 100% identical to SEQ ID NO:1. In embodiments, the siRNA comprises SEQ ID NO:2. In embodiments, the siRNA comprises a sequence that is at least 80% identical to SEQ ID NO:2. In embodiments, the siRNA comprises a sequence that is at least 85% identical to SEQ ID NO:2. In embodiments, the siRNA comprises a sequence that is at least 90% identical to SEQ ID NO:2. In embodiments, the siRNA comprises a sequence that is at least 95% identical to SEQ ID NO:2. In embodiments, the siRNA comprises a sequence that is at least 98% identical to SEQ ID NO:2. In embodiments, the siRNA comprises a sequence that is 100% identical to SEQ ID NO:2.

In embodiments, the KIAA0930 inhibitor is a GapmeR. In embodiments, the GapmeR comprises SEQ ID NO:3, SEQ ID NO:4, or SEQ ID NO:5. In embodiments, the GapmeR comprises SEQ ID NO:3. In embodiments, the GapmeR comprises a sequence that is at least 80% identical to SEQ ID NO:3. In embodiments, the GapmeR comprises a sequence that is at least 85% identical to SEQ ID NO:3. In embodiments, the GapmeR comprises a sequence that is at least 90% identical to SEQ ID NO:3. In embodiments, the GapmeR comprises a sequence that is at least 95% identical to SEQ ID NO:3. In embodiments, the GapmeR comprises a sequence that is at least 98% identical to SEQ ID NO:3. In embodiments, the GapmeR comprises a sequence that is 100% identical to SEQ ID NO:3. In embodiments, the GapmeR comprises SEQ ID NO:4. In embodiments, the GapmeR comprises a sequence that is at least 80% identical to SEQ ID NO:4. In embodiments, the GapmeR comprises a sequence that is at least 85% identical to SEQ ID NO:4. In embodiments, the GapmeR comprises a sequence that is at least 90% identical to SEQ ID NO:4. In embodiments, the GapmeR comprises a sequence that is at least 95% identical to SEQ ID NO:4. In embodiments, the GapmeR comprises a sequence that is at least 98% identical to SEQ ID NO:4. In embodiments, the GapmeR comprises a sequence that is 100% identical to SEQ ID NO:4. In embodiments, the GapmeR comprises SEQ ID NO:5. In embodiments, the GapmeR comprises a sequence that is at least 80% identical to SEQ ID NO:5. In embodiments, the GapmeR comprises a sequence that is at least 85% identical to SEQ ID NO:5. In embodiments, the GapmeR comprises a sequence that is at least 90% identical to SEQ ID NO:5. In embodiments, the GapmeR comprises a sequence that is at least 95% identical to SEQ ID NO:5. In embodiments, the GapmeR comprises a sequence that is at least 98% identical to SEQ ID NO:5. In embodiments, the GapmeR comprises a sequence that is 100% identical to SEQ ID NO:5.

In embodiments, the KIAA0930 inhibitor is a CRISPR Cas guide RNA (gRNA). In embodiments, the CRISPR Cas guide RNA is CRISPR Cas9 guide RNA. In embodiments, the CRISPR Cas9 guide RNA comprises SEQ ID NO:6, SEQ ID NO:7, or SEQ ID NO:10. In embodiments, the CRISPR Cas9 guide RNA comprises SEQ ID NO:6. In embodiments, the CRISPR Cas gRNA comprises a sequence that is at least 80% identical to SEQ ID NO:6. In embodiments, the CRISPR Cas gRNA comprises a sequence that is at least 85% identical to SEQ ID NO:6. In embodiments, the CRISPR Cas gRNA comprises a sequence that is at least 90% identical to SEQ ID NO:6. In embodiments, the CRISPR Cas gRNA comprises a sequence that is at least 95% identical to SEQ ID NO:6. In embodiments, the CRISPR Cas gRNA comprises a sequence that is at least 98% identical to SEQ ID NO:6. In embodiments, the CRISPR Cas gRNA comprises a sequence that is 100% identical to SEQ ID NO:6. In embodiments, the CRISPR Cas9 guide RNA comprises SEQ ID NO:7. In embodiments, the CRISPR Cas gRNA comprises a sequence that is at least 80% identical to SEQ ID NO:7. In embodiments, the CRISPR Cas gRNA comprises a sequence that is at least 85% identical to SEQ ID NO:7. In embodiments, the CRISPR Cas gRNA comprises a sequence that is at least 90% identical to SEQ ID NO:7. In embodiments, the CRISPR Cas gRNA comprises a sequence that is at least 95% identical to SEQ ID NO:7. In embodiments, the CRISPR Cas gRNA comprises a sequence that is at least 98% identical to SEQ ID NO:7. In embodiments, the CRISPR Cas gRNA comprises a sequence that is 100% identical to SEQ ID NO:7. In embodiments, the CRISPR Cas9 guide RNA comprises SEQ ID NO:10. In embodiments, the CRISPR Cas gRNA comprises a sequence that is at least 80% identical to SEQ ID NO:10. In embodiments, the CRISPR Cas gRNA comprises a sequence that is at least 85% identical to SEQ ID NO:10. In embodiments, the CRISPR Cas gRNA comprises a sequence that is at least 90% identical to SEQ ID NO:10. In embodiments, the CRISPR Cas gRNA comprises a sequence that is at least 95% identical to SEQ ID NO:10. In embodiments, the CRISPR Cas gRNA comprises a sequence that is at least 98% identical to SEQ ID NO:10. In embodiments, the CRISPR Cas gRNA comprises a sequence that is 100% identical to SEQ ID NO:10.

In embodiments, the CRISPR Cas guide RNA is CRISPR Cas 12 guide RNA.

In embodiments, the KIAA0930 inhibitor is a small molecule compound. In embodiments, the KIAA0930 inhibitor is a morpholinooligonucleotide.

In embodiments, the wasting syndrome is associated with cancer cachexia. In embodiments, the wasting syndrome is cancer cachexia. In embodiments, the subject has cancer and has a wasting syndrome comprising muscle atrophy with or without fat loss. In embodiments, the subject has cancer and has a wasting syndrome comprising muscle atrophy with fat loss. In embodiments, the subject has cancer and has a wasting syndrome comprising a muscle atrophy without fat loss.

In embodiments, the wasting syndrome is weight loss, fat loss, muscle atrophy, anorexia, asthenia, anemia, or a combination of two or more thereof. In embodiments, the wasting syndrome is weight loss. In embodiments, the wasting syndrome is fat loss. In embodiments, the wasting syndrome is muscle atrophy. In embodiments, the wasting syndrome is anorexia. In embodiments, the wasting syndrome is asthenia. In embodiments, the wasting syndrome is anemia. In embodiments, the wasting syndrome is muscle atropy with or without fat loss. In embodiments, the wasting syndrome is muscle atropy with fat loss. In embodiments, the wasting syndrome is muscle atropy without fat loss.

In embodiments, the subject does not respond to anti-cancer therapies. In embodiments, the subject does not respond to anti-inflammatory cytokine therapies. In embodiments, the anti-inflammatory cytokine therapy is IL-6, IL-1-alpha, TNF-alpha, TGF-beta, or a combination thereof. In embodiments, the subject does not respond to anti-cachexic therapies. In embodiments, the anti-cachexic therapy is an appetite stimulation, such as megestrol acetate, dronabinol, cyproheptadine, metoclopramide, cisapride, hydrazine sulfate, pentoxifylline, lisofylline, thalidomide, eicosapentaenoic acid, clenbuterol, growth hormone, or a combination thereof.

In embodiments, the cancer is pancreatic cancer, colorectal cancer, gastric cancer, head and neck cancer, or lung cancer. In embodiments, the cancer is pancreatic cancer, colorectal cancer, gastric cancer, head and neck cancer, or non-small cell lung cancer. In embodiments, the cancer is pancreatic cancer. In embodiments, the cancer is colorectal cancer. In embodiments, the cancer is gastric cancer. In embodiments, the cancer is head and neck cancer. In embodiments, the cancer is lung cancer. In embodiments, the cancer is non-small cell lung cancer.

In embodiments, the KIAA0930 inhibitor reduces or inhibits fat and muscle loss. In embodiments, the KIAA0930 inhibitor reduces or inhibits muscle atrophy. In embodiments, the KIAA0930 inhibitor reduces or inhibits anorexia. In embodiments, the KIAA0930 inhibitor reduces or inhibits asthenia. In embodiments, the KIAA0930 inhibitor reduces or inhibits anemia.

In embodiments, the KIAA0930 inhibitors as provided herein are administered as pharmaceutical compositions that further comprise one or more excipients or additives. In embodiments, the pharmaceutical compositions are administered by oral, lingual, sublingual, parenteral, rectal, topical, transdermal or pulmonary administration.

In embodiments, the disclosed methods further comprise administering to the subject platinum-based chemotherapy. In embodiments, the platinum-based chemotherapy comprises carboplatin, cisplatin, etoposide, a taxane, or any combination thereof. In embodiments, the pharmaceutical composition comprising the KIAA0930 inhibitor and the platinum-based chemotherapy are administered together. In embodiments, the pharmaceutical composition comprising the KIAA0930 inhibitor and the platinum-based chemotherapy are administered separately.

Pharmaceutical Compositions

Provided herein are pharmaceutical compositions for preventing or treating a wasting syndrome in a subject in need thereof. The disclosed pharmaceutical compositions comprise an effective amount of a KIAA0930 inhibitor and one or more excipients or additives.

In embodiments, the KIAA0930 inhibitor is a short-hairpin RNA (shRNA), a small interference RNA (siRNA), a piwi-interacting RNA (piRNA), a microRNA (miRNA), an antisense oligonucleotide, a CRISPR Cas guide RNA (gRNA), or a small molecule compound.

In embodiments, the KIAA0930 inhibitor is a shRNA. In embodiments, the shRNA comprises SEQ ID NO:8. In embodiments, the shRNA comprises a sequence that is at least 80% identical to SEQ ID NO:8. In embodiments, the shRNA comprises a sequence that is at least 85% identical to SEQ ID NO:8. In embodiments, the shRNA comprises a sequence that is at least 90% identical to SEQ ID NO:8. In embodiments, the shRNA comprises a sequence that is at least 95% identical to SEQ ID NO:8. In embodiments, the shRNA comprises a sequence that is at least 98% identical to SEQ ID NO:8. In embodiments, the shRNA comprises a sequence that is 100% identical to SEQ ID NO:8. In embodiments, the shRNA comprises SEQ ID NO:9. In embodiments, the shRNA comprises a sequence that is at least 80% identical to SEQ ID NO:9. In embodiments, the shRNA comprises a sequence that is at least 85% identical to SEQ ID NO:9. In embodiments, the shRNA comprises a sequence that is at least 90% identical to SEQ ID NO:9. In embodiments, the shRNA comprises a sequence that is at least 95% identical to SEQ ID NO:9. In embodiments, the shRNA comprises a sequence that is at least 98% identical to SEQ ID NO:9. In embodiments, the shRNA comprises a sequence that is 100% identical to SEQ ID NO:9.

In embodiments, the KIAA0930 inhibitor is a siRNA. In embodiments, the siRNA comprises SEQ ID NO:1. In embodiments, the siRNA comprises a sequence that is at least 80% identical to SEQ ID NO:1. In embodiments, the siRNA comprises a sequence that is at least 85% identical to SEQ ID NO:1. In embodiments, the siRNA comprises a sequence that is at least 90% identical to SEQ ID NO:1. In embodiments, the siRNA comprises a sequence that is at least 95% identical to SEQ ID NO:1. In embodiments, the siRNA comprises a sequence that is at least 98% identical to SEQ ID NO:1. In embodiments, the siRNA comprises a sequence that is 100% identical to SEQ ID NO:1. In embodiments, the siRNA comprises SEQ ID NO:2. In embodiments, the siRNA comprises a sequence that is at least 80% identical to SEQ ID NO:2. In embodiments, the siRNA comprises a sequence that is at least 85% identical to SEQ ID NO:2. In embodiments, the siRNA comprises a sequence that is at least 90% identical to SEQ ID NO:2. In embodiments, the siRNA comprises a sequence that is at least 95% identical to SEQ ID NO:2. In embodiments, the siRNA comprises a sequence that is at least 98% identical to SEQ ID NO:2. In embodiments, the siRNA comprises a sequence that is 100% identical to SEQ ID NO:2.

In embodiments, the antisense oligonucleotide is a GapmeR. In embodiments, the KIAA0930 inhibitor is a GapmeR. In embodiments, the GapmeR comprises SEQ ID NO:3, SEQ ID NO:4, or SEQ ID NO:5. In embodiments, the GapmeR comprises SEQ ID NO:3. In embodiments, the GapmeR comprises a sequence that is at least 80% identical to SEQ ID NO:3. In embodiments, the GapmeR comprises a sequence that is at least 85% identical to SEQ ID NO:3. In embodiments, the GapmeR comprises a sequence that is at least 90% identical to SEQ ID NO:3. In embodiments, the GapmeR comprises a sequence that is at least 95% identical to SEQ ID NO:3. In embodiments, the GapmeR comprises a sequence that is at least 98% identical to SEQ ID NO:3. In embodiments, the GapmeR comprises a sequence that is 100% identical to SEQ ID NO:3. In embodiments, the GapmeR comprises SEQ ID NO:4. In embodiments, the GapmeR comprises a sequence that is at least 80% identical to SEQ ID NO:4. In embodiments, the GapmeR comprises a sequence that is at least 85% identical to SEQ ID NO:4. In embodiments, the GapmeR comprises a sequence that is at least 90% identical to SEQ ID NO:4. In embodiments, the GapmeR comprises a sequence that is at least 95% identical to SEQ ID NO:4. In embodiments, the GapmeR comprises a sequence that is at least 98% identical to SEQ ID NO:4. In embodiments, the GapmeR comprises a sequence that is 100% identical to SEQ ID NO:4. In embodiments, the GapmeR comprises SEQ ID NO:5. In embodiments, the GapmeR comprises a sequence that is at least 80% identical to SEQ ID NO:5. In embodiments, the GapmeR comprises a sequence that is at least 85% identical to SEQ ID NO:5. In embodiments, the GapmeR comprises a sequence that is at least 90% identical to SEQ ID NO:5. In embodiments, the GapmeR comprises a sequence that is at least 95% identical to SEQ ID NO:5. In embodiments, the GapmeR comprises a sequence that is at least 98% identical to SEQ ID NO:5. In embodiments, the GapmeR comprises a sequence that is 100% identical to SEQ ID NO:5.

In embodiments, the KIAA0930 inhibitor is a CRISPR Cas guide RNA (gRNA). In embodiments, the CRISPR Cas guide RNA is CRISPR Cas9 guide RNA. In embodiments, the CRISPR Cas9 guide RNA comprises SEQ ID NO:6, SEQ ID NO:7, or SEQ ID NO:10. In embodiments, the CRISPR Cas9 guide RNA comprises SEQ ID NO:6. In embodiments, the CRISPR Cas gRNA comprises a sequence that is at least 80% identical to SEQ ID NO:6. In embodiments, the CRISPR Cas gRNA comprises a sequence that is at least 85% identical to SEQ ID NO:6. In embodiments, the CRISPR Cas gRNA comprises a sequence that is at least 90% identical to SEQ ID NO:6. In embodiments, the CRISPR Cas gRNA comprises a sequence that is at least 95% identical to SEQ ID NO:6. In embodiments, the CRISPR Cas gRNA comprises a sequence that is at least 98% identical to SEQ ID NO:6. In embodiments, the CRISPR Cas gRNA comprises a sequence that is 100% identical to SEQ ID NO:6. In embodiments, the CRISPR Cas9 guide RNA comprises SEQ ID NO:7. In embodiments, the CRISPR Cas gRNA comprises a sequence that is at least 80% identical to SEQ ID NO:7. In embodiments, the CRISPR Cas gRNA comprises a sequence that is at least 85% identical to SEQ ID NO:7. In embodiments, the CRISPR Cas gRNA comprises a sequence that is at least 90% identical to SEQ ID NO:7. In embodiments, the CRISPR Cas gRNA comprises a sequence that is at least 95% identical to SEQ ID NO:7. In embodiments, the CRISPR Cas gRNA comprises a sequence that is at least 98% identical to SEQ ID NO:7. In embodiments, the CRISPR Cas gRNA comprises a sequence that is 100% identical to SEQ ID NO:7. In embodiments, the CRISPR Cas9 guide RNA comprises SEQ ID NO:10. In embodiments, the CRISPR Cas gRNA comprises a sequence that is at least 80% identical to SEQ ID NO:10. In embodiments, the CRISPR Cas gRNA comprises a sequence that is at least 85% identical to SEQ ID NO:10. In embodiments, the CRISPR Cas gRNA comprises a sequence that is at least 90% identical to SEQ ID NO:10. In embodiments, the CRISPR Cas gRNA comprises a sequence that is at least 95% identical to SEQ ID NO:10. In embodiments, the CRISPR Cas gRNA comprises a sequence that is at least 98% identical to SEQ ID NO:10. In embodiments, the CRISPR Cas gRNA comprises a sequence that is 100% identical to SEQ ID NO:10.

In embodiments, the CRISPR Cas guide RNA is CRISPR Cas 12 guide RNA.

In embodiments, the KIAA0930 inhibitor is a small molecule compound. In embodiments, the KIAA0930 inhibitor is a morpholinooligonucleotide.

In embodiments, the pharmaceutical compositions provided herein are suitable for oral, lingual, sublingual, parenteral, rectal, topical, transdermal or pulmonary administration.

KIAA0930 Inhibitors

Provided herein is a KIAA0930 inhibitor for treating or preventing a wasting syndrome. In embodiments, the KIAA0930 inhibitor is a shRNA, a small interference RNA (siRNA), a piwi-interacting RNA (piRNA), a microRNA (miRNA), an antisense oligonucleotide, a CRISPR Cas guide RNA (gRNA), or a small molecule compound.

In embodiments, the KIAA0930 inhibitor is a short-hairpin RNA (shRNA). In embodiments, the shRNA comprises SEQ ID NO:8. In embodiments, the shRNA comprises a sequence that is at least 80% identical to SEQ ID NO:8. In embodiments, the shRNA comprises a sequence that is at least 85% identical to SEQ ID NO:8. In embodiments, the shRNA comprises a sequence that is at least 90% identical to SEQ ID NO:8. In embodiments, the shRNA comprises a sequence that is at least 95% identical to SEQ ID NO:8. In embodiments, the shRNA comprises a sequence that is at least 98% identical to SEQ ID NO:8. In embodiments, the shRNA comprises a sequence that is 100% identical to SEQ ID NO:8. In embodiments, the shRNA comprises SEQ ID NO:9. In embodiments, the shRNA comprises a sequence that is at least 80% identical to SEQ ID NO:9. In embodiments, the shRNA comprises a sequence that is at least 85% identical to SEQ ID NO:9. In embodiments, the shRNA comprises a sequence that is at least 90% identical to SEQ ID NO:9. In embodiments, the shRNA comprises a sequence that is at least 95% identical to SEQ ID NO:9. In embodiments, the shRNA comprises a sequence that is at least 98% identical to SEQ ID NO:9. In embodiments, the shRNA comprises a sequence that is 100% identical to SEQ ID NO:9.

In embodiments, the KIAA0930 inhibitor is a small interference RNA (siRNA). In embodiments, the siRNA comprises SEQ ID NO:1. In embodiments, the siRNA comprises a sequence that is at least 80% identical to SEQ ID NO:1. In embodiments, the siRNA comprises a sequence that is at least 85% identical to SEQ ID NO:1. In embodiments, the siRNA comprises a sequence that is at least 90% identical to SEQ ID NO:1. In embodiments, the siRNA comprises a sequence that is at least 95% identical to SEQ ID NO:1. In embodiments, the siRNA comprises a sequence that is at least 98% identical to SEQ ID NO:1. In embodiments, the siRNA comprises a sequence that is 100% identical to SEQ ID NO:1. In embodiments, the siRNA comprises SEQ ID NO:2. In embodiments, the siRNA comprises a sequence that is at least 80% identical to SEQ ID NO:2. In embodiments, the siRNA comprises a sequence that is at least 85% identical to SEQ ID NO:2. In embodiments, the siRNA comprises a sequence that is at least 90% identical to SEQ ID NO:2. In embodiments, the siRNA comprises a sequence that is at least 95% identical to SEQ ID NO:2. In embodiments, the siRNA comprises a sequence that is at least 98% identical to SEQ ID NO:2. In embodiments, the siRNA comprises a sequence that is 100% identical to SEQ ID NO:2.

In embodiments, the antisense oligonucleotide is a GapmeR. In embodiments, the KIAA0930 inhibitor is a GapmeR. In embodiments, the GapmeR comprises SEQ ID NO:3, SEQ ID NO:4, or SEQ ID NO:5. In embodiments, the GapmeR comprises SEQ ID NO:3. In embodiments, the GapmeR comprises a sequence that is at least 80% identical to SEQ ID NO:3. In embodiments, the GapmeR comprises a sequence that is at least 85% identical to SEQ ID NO:3. In embodiments, the GapmeR comprises a sequence that is at least 90% identical to SEQ ID NO:3. In embodiments, the GapmeR comprises a sequence that is at least 95% identical to SEQ ID NO:3. In embodiments, the GapmeR comprises a sequence that is at least 98% identical to SEQ ID NO:3. In embodiments, the GapmeR comprises a sequence that is 100% identical to SEQ ID NO:3. In embodiments, the GapmeR comprises SEQ ID NO:4. In embodiments, the GapmeR comprises a sequence that is at least 80% identical to SEQ ID NO:4. In embodiments, the GapmeR comprises a sequence that is at least 85% identical to SEQ ID NO:4. In embodiments, the GapmeR comprises a sequence that is at least 90% identical to SEQ ID NO:4. In embodiments, the GapmeR comprises a sequence that is at least 95% identical to SEQ ID NO:4. In embodiments, the GapmeR comprises a sequence that is at least 98% identical to SEQ ID NO:4. In embodiments, the GapmeR comprises a sequence that is 100% identical to SEQ ID NO:4. In embodiments, the GapmeR comprises SEQ ID NO:5. In embodiments, the GapmeR comprises a sequence that is at least 80% identical to SEQ ID NO:5. In embodiments, the GapmeR comprises a sequence that is at least 85% identical to SEQ ID NO:5. In embodiments, the GapmeR comprises a sequence that is at least 90% identical to SEQ ID NO:5. In embodiments, the GapmeR comprises a sequence that is at least 95% identical to SEQ ID NO:5. In embodiments, the GapmeR comprises a sequence that is at least 98% identical to SEQ ID NO:5. In embodiments, the GapmeR comprises a sequence that is 100% identical to SEQ ID NO:5.

In embodiments, the KIAA0930 inhibitor is a CRISPR Cas guide RNA (gRNA). In embodiments, the CRISPR Cas guide RNA is CRISPR Cas9 guide RNA. In embodiments, the CRISPR Cas9 guide RNA comprises SEQ ID NO:6, SEQ ID NO:7, or SEQ ID NO:10. In embodiments, the CRISPR Cas9 guide RNA comprises SEQ ID NO:6. In embodiments, the CRISPR Cas gRNA comprises a sequence that is at least 80% identical to SEQ ID NO:6. In embodiments, the CRISPR Cas gRNA comprises a sequence that is at least 85% identical to SEQ ID NO:6. In embodiments, the CRISPR Cas gRNA comprises a sequence that is at least 90% identical to SEQ ID NO:6. In embodiments, the CRISPR Cas gRNA comprises a sequence that is at least 95% identical to SEQ ID NO:6. In embodiments, the CRISPR Cas gRNA comprises a sequence that is at least 98% identical to SEQ ID NO:6. In embodiments, the CRISPR Cas gRNA comprises a sequence that is 100% identical to SEQ ID NO:6. In embodiments, the CRISPR Cas9 guide RNA comprises SEQ ID NO:7. In embodiments, the CRISPR Cas gRNA comprises a sequence that is at least 80% identical to SEQ ID NO:7. In embodiments, the CRISPR Cas gRNA comprises a sequence that is at least 85% identical to SEQ ID NO:7. In embodiments, the CRISPR Cas gRNA comprises a sequence that is at least 90% identical to SEQ ID NO:7. In embodiments, the CRISPR Cas gRNA comprises a sequence that is at least 95% identical to SEQ ID NO:7. In embodiments, the CRISPR Cas gRNA comprises a sequence that is at least 98% identical to SEQ ID NO:7. In embodiments, the CRISPR Cas gRNA comprises a sequence that is 100% identical to SEQ ID NO:7. In embodiments, the CRISPR Cas9 guide RNA comprises SEQ ID NO:10. In embodiments, the CRISPR Cas gRNA comprises a sequence that is at least 80% identical to SEQ ID NO:10. In embodiments, the CRISPR Cas gRNA comprises a sequence that is at least 85% identical to SEQ ID NO:10. In embodiments, the CRISPR Cas gRNA comprises a sequence that is at least 90% identical to SEQ ID NO:10. In embodiments, the CRISPR Cas gRNA comprises a sequence that is at least 95% identical to SEQ ID NO:10. In embodiments, the CRISPR Cas gRNA comprises a sequence that is at least 98% identical to SEQ ID NO:10. In embodiments, the CRISPR Cas gRNA comprises a sequence that is 100% identical to SEQ ID NO:10.

In embodiments, the CRISPR Cas guide RNA is CRISPR Cas 12 guide RNA.

In embodiments, the KIAA0930 inhibitor is a small molecule compound. In embodiments, the KIAA0930 inhibitor is a morpholinooligonucleotide.

Tables 1-6

TABLE 1
Sequences of siRNA targeting KIAA0930

| | Sequence | |
|---|---|---|
| | (Sense strand 5′→3′) | SEQ ID |
| siRNA1 | GCGUACUCCGGCAGCGAAA | SEQ ID NO: 1 |
| siRNA2 | GCUUCAAGGAUGACCGCAU | SEQ ID NO: 2 |

TABLE 2
Sequences of GapmeR targeting KIAA0930

| | Sequence (5′→3′)* | SEQ ID |
|---|---|---|
| GapmeR1 | AACACACGCCGTCAAA | SEQ ID NO: 3 |
| GapmeR2 | ACGTAGGTTAAGTGTG | SEQ ID NO: 4 |
| GapmeR3 | ACTAGAGCAAGAGGAC | SEQ ID NO: 5 |

*Sequence contains phosphorothioate backbone. Nucleotides shown in bold-underline are locked nucleic acids. Other nucleotides are deoxyribonucleotides.

TABLE 3
Sequences of sgRNA targeting KIAA0930

| | Sequence (5′→3′) | |
|---|---|---|
| gRNA1 | AAAAGCATGTCGTCCTGCCG | SEQ ID NO: 6 |
| gRNA2 | GGGAGACCCTGACATCGACT | SEQ ID NO: 7 |

TABLE 4
Sequences of shRNA targeting KIAA0930

| | Sequence | |
|---|---|---|
| | (5′→3′) Sense-Loop-Antisense | |
| shRNA1 | GACATTCACATCCATAAGAAGTTGGATCCAA | SEQ ID NO: 8 |
| | CTTCTTATGGATGTGAATGTC | |
| shRNA2 | GCAACATGGAGTTTGTGCGCATTGGATCCAA | SEQ ID NO: 9 |
| | TGCGCACAAACTCCATGTTGC | |

TABLE 5
Sequences of guideRNA targeting KIAA0930

| | Sequence (5′→3′) | |
|---|---|---|
| gRNA3 | GCGGTGTGCACACGTGCTGA | SEQ ID NO: 10 |

TABLE 6
Primers

| | | | |
|---|---|---|---|
| KIAA0930 | FWD | TCTTTCAGGGCTCCATCCGCTA | SEQ ID NO: 11 |
| KIAA0930 | REV | GCGCACAAACTCCATGTTGCTG | SEQ ID NO: 12 |
| β-Actin | FWD | CACCATTGGCAATGAGCGGTTC | SEQ ID NO: 13 |
| β-Actin | REV | AGGTCTTTGCGGATGTCCACGT | SEQ ID NO: 14 |

### Embodiments N1-N20

Embodiment N1. A method of preventing or treating a wasting syndrome in a subject in need thereof, the method comprising administering to the subject an effective amount of a KIAA0930 inhibitor.

Embodiment N2. The method of Embodiment N1, wherein the KIAA0930 inhibitor is a short-hairpin RNA, a small interference RNA, a piwi-interacting RNA, a microRNA, a CRISPR Cas guide RNA, an antisense oligonucleotide, or a small molecule compound.

Embodiment N3. The method of Embodiment N1, wherein the KIAA0930 inhibitor is: (a) a short-hairpin RNA comprising SEQ ID NO:8; (b) a short-hairpin RNA comprising SEQ ID NO:9; (c) a small-interference RNA comprising SEQ ID NO:1; (d) a small-interference RNA comprising SEQ ID NO:2; (e) a GapmeR comprising SEQ ID NO:3; (f) a GapmeR comprising SEQ ID NO:4; (g) a GapmeR comprising SEQ ID NO:5; (h) a CRISPR Cas9 guide RNA comprising SEQ ID NO:6; (i) a CRISPR Cas9 guide RNA comprising SEQ ID NO:7; or (j) a CRISPR Cas9 guide RNA comprising SEQ ID NO:10.

Embodiment N4. The method of Embodiment N1, wherein the KIAA0930 inhibitor is a GapmeR.

Embodiment N5. The method of Embodiment N1, wherein the KIAA0930 inhibitor is a CRISPR Cas9 guide RNA.

Embodiment N6. The method of Embodiment N1, wherein the KIAA0930 inhibitor is a morpholinooligonucleotide.

Embodiment N7. The method of Embodiment N1, wherein the KIAA0930 inhibitor is a CRISPR Cas 12 guide RNA.

Embodiment N8. The method of any one of Embodiments N1 to N7, wherein the wasting syndrome is cancer cachexia.

Embodiment N9. The method of Embodiment N8, wherein the cancer is pancreatic cancer, colorectal cancer, gastric cancer, head and neck cancer, or lung cancer.

Embodiment N10. The method of any one of Embodiments N1 to N9, wherein the wasting syndrome is weight loss, fat loss, muscle atrophy, anorexia, asthenia, or anemia.

Embodiment N11. The method of any one of Embodiments N1 to N9, wherein the wasting syndrome is muscle atrophy.

Embodiment N12. The method of any one of Embodiments N1 to N9, wherein the KIAA0930 inhibitor reduces or inhibits fat and muscle loss; reduces or inhibits muscle atrophy; reduces or inhibits anorexia; reduces or inhibits asthenia; reduces or inhibits anemia; or a combination of two or more thereof.

Embodiment N13. The method of any one of Embodiments Ni to N12, wherein administering is oral, lingual, sublingual, parenteral, rectal, topical, transdermal, or pulmonary.

Embodiment N14. The method of any one of Embodiments Ni to N13, further comprising administering to the subject an effective amount of an anti-cancer agent.

Embodiment N15. A pharmaceutical composition comprising a KIAA0930 inhibitor and a pharmaceutically acceptable excipient.

Embodiment N16. The pharmaceutical composition of Embodiment N15, wherein the KIAA0930 inhibitor is a short-hairpin RNA, a small interference RNA, a piwi-interacting RNA, a microRNA, a CRISPR Cas guide RNA, an antisense oligonucleotide, or a small molecule compound.

Embodiment N17 The pharmaceutical composition of Embodiment N15 wherein the KIAA0930 inhibitor is: (a) a short-hairpin RNA comprising SEQ ID NO:8; (b) a short-hairpin RNA comprising SEQ ID NO:9; (c) a small-interference RNA comprising SEQ ID NO:1; (d) a small-interference RNA comprising SEQ ID NO:2; (e) a GapmeR comprising SEQ ID NO:3; (f) a GapmeR comprising SEQ ID NO:4; (g) a GapmeR comprising SEQ ID NO:5; (h) a CRISPR Cas9 guide RNA comprising SEQ ID NO:6; (i) a CRISPR Cas9 guide RNA comprising SEQ ID NO:7; or (j) a CRISPR Cas9 guide RNA comprising SEQ ID NO:10.

Embodiment N18. The pharmaceutical composition of Embodiment N15, wherein the KIAA0930 inhibitor is a GapmeR or a morpholinooligonucleotide.

Embodiment N19. The pharmaceutical composition of Embodiment N15, wherein the KIAA0930 inhibitor is CRISPR Cas9 guide RNA or a CRISPR Cas 12 guide RNA.

Embodiment N20. A KIAA0930 inhibitor, wherein the KIAA0930 inhibitor is: (a) a short-hairpin RNA comprising SEQ ID NO:8; (b) a short-hairpin RNA comprising SEQ ID NO:9; (c) a small-interference RNA comprising SEQ ID NO:1; (d) a small-interference RNA comprising SEQ ID NO:2; (e) a GapmeR comprising SEQ ID NO:3; (f) a GapmeR comprising SEQ ID NO:4; (g) a GapmeR comprising SEQ ID NO:5; (h) a CRISPR Cas9 guide RNA comprising SEQ ID NO:6; (i) a CRISPR Cas9 guide RNA comprising SEQ ID NO:7; or (j) a CRISPR Cas9 guide RNA comprising SEQ ID NO:10.

Embodiments 1-51

Embodiment 1. A method of preventing or treating a wasting syndrome, in a subject in need thereof, wherein the method comprises administering to the subject an effective amount of a KIAA0930 inhibitor.

Embodiment 2. The method of Embodiment 1, wherein the inhibitor suppresses or reduces expression of a KIAA0930 protein.

Embodiment 3. The method of Embodiment 1 or embodiment 2, wherein the KIAA0930 inhibitor is a short-hairpin RNA (shRNA), a small interference RNA (siRNA), a piwi-interacting RNA (piRNA), a microRNA (miRNA), a CRISPR Cas guide RNA (gRNA), an antisense oligonucleotide, or a small molecule compound.

Embodiment 4. The method of Embodiment 3, wherein the short-hairpin RNA (shRNA) comprises SEQ ID NO:8.

Embodiment 5. The method of Embodiment 3, wherein the short-hairpin RNA (shRNA) comprises SEQ ID NO:9.

Embodiment 6. The method of Embodiment 3, wherein the small-interference RNA (siRNA) comprises SEQ ID NO:1 or SEQ ID NO:2.

Embodiment 7. The method of Embodiment 3, wherein the antisense oligonucleotide is a GapmeR or a morpholinooligonucleotide.

Embodiment 8. The method of Embodiment 7, wherein the GapmeR comprises SEQ ID NO:3, SEQ ID NO:4, or SEQ ID NO:5.

Embodiment 9. The method of Embodiment 3, wherein the CRISPR Cas guide RNA is CRISPR Cas9 guide RNA.

Embodiment 10. The method of Embodiment 8, wherein the CRISPR Cas9 guide RNA comprises SEQ ID NO:6, SEQ ID NO:7, or SEQ ID NO:10.

Embodiment 11. The method of Embodiment 3, wherein the CRISPR Cas guide RNA is CRISPR Cas 12 guide RNA.

Embodiment 12. The method of any one of Embodiments 1-10, wherein the wasting syndrome is associated with cancer cachexia.

Embodiment 13. The method of Embodiment 11, wherein the wasting syndrome is weight loss.

Embodiment 14. The method of Embodiment 11, wherein the wasting syndrome is fat loss.

Embodiment 15. The method of Embodiment 11, wherein the wasting syndrome is muscle atrophy.

Embodiment 16. The method of Embodiment 11, wherein the wasting syndrome is anorexia.

Embodiment 17. The method of Embodiment 11, wherein the wasting syndrome is asthenia.

Embodiment 18. The method of Embodiment 11, wherein the wasting syndrome is anemia.

Embodiment 19. The method of any one of Embodiments 1-17, wherein the subject does not respond to anti-cancer therapies.

Embodiment 20. The method of any one of Embodiments 1-19, wherein the subject does not respond to anti-inflammatory cytokine therapies.

Embodiment 21. The method of Embodiment 19, wherein the subject does not respond to anti-cachexic therapies.

Embodiment 22. The method of any one of Embodiments 18-20, wherein the cancer is pancreatic cancer, colorectal cancer, gastric cancer, head and neck cancer, or lung cancer.

Embodiment 23. The method of any one of Embodiments 1-21, wherein the KIAA0930 inhibitor reduces or inhibits fat and muscle loss.

Embodiment 24. The method of any one of Embodiments 1-21, wherein the KIAA0930 inhibitor reduces or inhibits muscle atrophy.

Embodiment 25. The method of any one of Embodiments 1-21, wherein the KIAA0930 inhibitor reduces or inhibits anorexia.

Embodiment 26. The method of any one of Embodiments 1-21, wherein the KIAA0930 inhibitor reduces or inhibits asthenia.

Embodiment 27. The method of any one of Embodiments 1-21, wherein the KIAA0930 inhibitor reduces or inhibits anemia.

Embodiment 28. The method of any one of Embodiments 1-26, wherein the KIAA0930 inhibitor is administered as a pharmaceutical composition that further comprises a pharmaceutically acceptable excipient.

Embodiment 29. The method of Embodiment 27, wherein the pharmaceutical composition is administered by oral, lingual, sublingual, parenteral, rectal, topical, transdermal, or pulmonary administration.

Embodiment 30. The method of any one of Embodiments 1-29, wherein the method further comprises administering to the subject an anti-cancer agent.

Embodiment 31. The method of Embodiment 30, wherein the pharmaceutical composition and the anti-cancer agent are administered together.

Embodiment 32. The method of Embodiment 30, wherein the pharmaceutical composition and the anti-cancer agent are administered separately.

Embodiment 33. A pharmaceutical composition for preventing or treating a wasting syndrome in a subject in need thereof, wherein the pharmaceutical composition comprises an effective amount of a KIAA0930 inhibitor and a pharmaceutically acceptable excipient.

Embodiment 34. The pharmaceutical composition of Embodiment 33, wherein the KIAA0930 inhibitor is a short-hairpin RNA (shRNA), a small interference RNA (siRNA), a piwi-interacting RNA (piRNA), a microRNA (miRNA), a CRISPR Cas guide RNA (gRNA), an antisense oligonucleotide, or a small molecule compound.

Embodiment 35. The pharmaceutical composition of Embodiment 34, wherein the short-hairpin RNA (shRNA) comprises SEQ ID NO:8.

Embodiment 36. The pharmaceutical composition of Embodiment 34, wherein the short-hairpin RNA (shRNA) comprises SEQ ID NO:9.

Embodiment 37. The pharmaceutical composition of Embodiment 34, wherein the small-interference RNA (siRNA) comprises SEQ ID NO:1 or SEQ ID NO:2.

Embodiment 38. The pharmaceutical composition of Embodiment 34, wherein the antisense oligonucleotide comprises a GapmeR or a morpholinooligonucleotide.

Embodiment 39. The pharmaceutical composition of Embodiment 38, wherein the GapmeR comprises SEQ ID NO:3, SEQ ID NO:4, or SEQ ID NO:5.

Embodiment 40. The pharmaceutical composition of Embodiment 34, wherein the CRISPR Cas guide RNA is CRISPR Cas9 guide RNA.

Embodiment 41. The pharmaceutical composition of Embodiment 40, wherein the CRISPR Cas9 guide RNA comprises SEQ ID NO:6, SEQ ID NO:7, or SEQ ID NO:10.

Embodiment 42. The pharmaceutical composition of Embodiment 34, wherein the CRISPR Cas guide RNA is CRISPR Cas 12 guide RNA.

Embodiment 43. The pharmaceutical composition of any one of Embodiments 33-42, wherein the pharmaceutical composition is suitable for oral, lingual, sublingual, parenteral, rectal, topical, transdermal, or pulmonary administration.

Embodiment 44. A KIAA0930 inhibitor, wherein the KIAA0930 inhibitor is a short-hairpin RNA (shRNA) that comprises SEQ ID NO:8.

Embodiment 45. A KIAA0930 inhibitor, wherein the KIAA0930 inhibitor is a short-hairpin RNA (shRNA) that comprises SEQ ID NO:9.

Embodiment 46. A KIAA0930 inhibitor, wherein the KIAA0930 inhibitor is a small-interference RNA (siRNA) that comprises SEQ ID NO:1 or SEQ ID NO:2.

Embodiment 47. A KIAA0930 inhibitor, wherein the KIAA0930 inhibitor is a GapmeR that comprises SEQ ID NO:3, SEQ ID NO:4, or SEQ ID NO:5.

Embodiment 48. A KIAA0930 inhibitor, wherein the KIAA0930 inhibitor is a CRISPR Cas guide RNA.

Embodiment 49. The KIAA0930 inhibitor of Embodiment 48, wherein the CRISPR Cas guide RNA is CRISPR Cas9 guide RNA.

Embodiment 50. The KIAA0930 inhibitor of Embodiment 49, wherein the CRISPR Cas9 guide RNA comprises SEQ ID NO:6, SEQ ID NO:7, or SEQ ID NO:10.

Embodiment 51. The KIAA0930 inhibitor of Embodiment 48, wherein the CRISPR Cas guide RNA is CRISPR Cas 12 guide RNA.

### Example

To explore novel targets for cancer cachexia (CC), we searched molecules that are upregulated in cancers in clinical microarray datasets and have not been previously investigated. We found that the uncharacterized transcript, KIAA0930 (also known as chromosome 22 open reading frame 9: C22orf9) confers cachexic phenotype in various cancer cell lines through cytokine-independent mechanisms.

We stably knocked down KIAA0930 in five pancreatic cancer cell lines, two colorectal cancer cell lines, and one gastric cancer cell line by lentiviral shRNA transduction. After confirmation of more than 50% knockdown, we collected conditioned medium (CM) from these cells. The murine myoblasts, C2C12 cells were differentiated into myotubes for 5 days in 2% horse serum. The myotubes were then treated with the CM or culture medium (non-conditioned medium: NCM) for 2 days, and the myotube diameter was measured after staining with myosin heavy chain (WIC). The CM from control cell lines caused a decrease in myotube diameter, compared to NCM. CM from KIAA0930 knockdown cells suppressed muscle atrophy, as represented by the increase in diameter. Importantly, this effect was observed in all cell lines we tested. Since CC is thought to be caused by pro-inflammatory cytokines secreted from cancer cells, we measured several cytokines, including TGFbeta, IL-1, 6, 8, and MCP-1 in CM. Cytokine profiles of CM from cancer cell lines were different from one another, and knockdown of KIAA0930 did not lead to consistent changes in cytokine secretion. These data indicate that KIAA0930 affects a cachexic phenotype through cytokine-independent mechanisms. Knockdown of KIAA0930 did not show a consistent proliferative effect among cell lines. In order to test whether anti-cachexic effect is also observed in a preclinical setting, we conducted orthotopic xenograft assay using PANC-1 cells with KIAA0930 being knocked down. The control PANC-1-bearing mice showed a significant decrease in a weight and cross section area of tibialis anterior (TA) muscle, compared to normal control (saline injection). The knockdown of KIAA0930 significantly ameliorated TA muscle atrophy, without a change in tumor weight. Collectively, these data show that KIAA0930 confers cachexic phenotype in multiple types of cancers.

We utilized lentiviral particles encoding short-hairpin RNA targeted to KIAA0930, and nucleic acid drugs, including small interference RNA, GapmeR, and morpholino oligonucleotides to reduce the expression of KIAA0930.

Cell Line Culture and Transfection. The human pancreatic cancer (PaCa) cell lines, PANC-1, Capan-2, CFPAC-1, Mia PaCa-2 and Panc 02.13, the human colorectal cancer (CRC) cell line, HCT116 and HT29, the human tongue cancer (TC) cell line, CAL 27 and SCC-15, the lung cancer cell line, A-427, the mouse myoblast cell line, C2C12, and the human colon fibroblast cell line, CCD-18co were purchased from American Type Culture Collection (Manassas, VA). The human gastric cancer (GC) cell line, MKN45 was purchased from Japanese Collection of Research Bioresources Cell Bank (Osaka, Japan). Primary human pancreatic and hepatic stellate cells (HPaSteC and HHSteC, respectively) were purchased from ScienCell (Carlsbad, CA).

PANC-1, CAL 27 and C2C12 cells were maintained in DME high-glucose medium (Irvine Scientific; Santa Ana, CA) supplemented with 10% Fetal bovine serum (FBS, from Cytiva; Marlborough, MA). Capan-2, HCT116, and HT29 cell lines were maintained in McCoy's 5A medium (Corning; Corning, NY) supplemented with 10% FBS. CFPAC-1 cells were maintained in Iscove's Modified Dulbecco's Medium (Thermo Fisher Scientific; Pittsburgh, PA) supplemented with 10% FBS. Mia PaCa-2 cell line was maintained in DME medium (ATCC) supplemented with 10% FBS and 2.5% horse serum (HS, from Thermo Fisher Scientific). Panc 02.13 cell line was maintained in RPMI-1640 medium (ATCC) supplemented with 15% FBS and 10 U/ml human insulin (MP Biomedicals; Solon, OH). SCC-15 cells line was maintained in DMEM/F-12 medium (Thermo Fisher Scientific) suppremented with 10% FBS and 400 ng/ml hydrocortisone. MKN45 cell line was maintained in RPMI-1640 medium (Irvine Scientific) supplemented with 10% FBS. A-427 and CCD-18co cell lines were maintained in EMEM medium (ATCC) supplemented with 10% FBS. HPaSteC and HHSteC were maintained in stellate cell medium supplemented with stellate cell growth supplement and 2% FBS (ScienCell) and the cells were transduced with pLV-hTERT-IRES-hygro lentivirus to establish immortalized cells (hTERT-HPaSteC and hTERT-HHSteC, respectively, see below).

Cells were transfected with nontargeting siRNA (NTsiRNA), human KIAA0930 targeting siRNA (Horizon Discovery; Cambridge, United Kingdom), nontargeting GapmeR, GapmeR targeted to KIAA0930 (QIAGEN; Germantown, MD), nontargeted single guide RNA (sgRNA), or KIAA0930 targeting sgRNA (IDT Integrated DNA Technologies; Coralville, IA) at 50 nM (for hTERT-HHSteC, 25 nM) for 24h using Lipofectamine 3000 (Thermo Fisher Scientific) according to the manufacturer's protocol. The sequences of these siRNA, GapmeR, and sgRNA are shown in Table 1, 2, and 3, respectively.

Lentiviral Packaging and Transduction. The palindromic oligos of nontargeted shRNA and KIAA0930 shRNA (shRNA1 and shRNA2: Table 4) were annealed and inserted into pLV-hU6-EF1a-GFP-Bsd shRNA vector (Biosettia; San Diego, CA) according to the manufacturer's instructions. For stable expression of Cas9-guide RNA, guideRNA targeted to KIAA0930 (gRNA3: Table 5) oligonucleotide duplex was inserted into lentiGuide-puro (Addgene; Watertown, MA) according to Sanjana et al, Nat Methods. 2014; 11(8):783-784. For immortalization of primary stellate cells, a pLV-hTERT-IRES-hygro lentivector, which is a gift from Tobias Meyer (Addgene plasmid #85140; http://n2t.net/addgene:85140; RRID:Addgene 85140; Watertown, MA) To produce lentiviral particles, 293T cells were transfected with these lentiviral vectors and packaging plasmids using Lipofectamine 3000 reagent (Thermo Fisher Scientific). The culture supernatants were collected and concentrated using Speedy Lentivirus Purification reagent (Applied Biological Materials, Inc.; Richmond, British Columbia, Canada). The viral titer was determined using 293T cells as described by Barczak et al, Molecular Biotechnology. 2014; 57(2):195-200. Cancer cells were transduced for 24 hr at MOI's of 10 in the presence of 6 μg/mL polybrene and selected with hygromycin for immortalization, blasticidin for shRNA, or puromycin for guideRNA.

Preparation of Conditional Medium from Cancer Cells. Cells were seeded in a 12-well plate at 4×10⁵ cells/well (CRC cell lines) or 2×10⁵ cells/well (other cells). One-day after, the cells were washed and cultured in 0.6 ml/well of growth medium containing FBS without growth supplements and/or additives. After 3 days, cell culture supernatants were collected, centrifuged at 300×g for 5 min, and stored at −80° C. as a conditioned medium (CM). A growth medium was also incubated without cells as a non-conditioned medium (NCM). To collect CM from transiently transfected cells, cells were seeded in a 12-well plate at 2×10 5 cells/well (CRC cell lines) or 1.5×10⁵ cells/well (other cells) and transfected one-day after seeding. Then cells were washed and cultured as described above.

Treatment of stellate cells and fibroblasts with cancer CM. hTERT-HPaSteC, hTERT-HHSteC, and CCD-18co cells were seeded, cultured, and transfected with siRNA as described above. Then cells were washed twice with growth medium supplemented with FBS without growth supplements and cultured in 10% cancer CM containing medium for 3 days. For no cell control, the medium containing 10% cancer CM without cells was cultured for 3 days, and CM was collected as describe previously.

RNA Analysis. Total RNA was extracted using Direct-zol RNA miniprep. (Zymo Research; Irvine, CA). cDNA was synthesized using PrimeScript RT reagent kit (TaKaRa Bio Inc.; San Jose, CA). Messenger RNA expression levels were determined using real-time RT-PCR. Real-time RT-PCR was performed with CFX96 real-time PCR Detection System, using the iQ SYBR Green supermix reagent (Bio-Rad; Hercules, CA). Messenger RNA expression was normalized to B-actin. Primer sequences are shown in Table 6.

Western Blotting. Cell monolayers were washed with ice-cold phosphate buffered saline (PBS), and cells were harvested in ice-cold lysis buffer (Cell Signaling Technology; Danvers, MA) supplemented with 1 mM phenylmethylsulfonyl fluoride. Lysates were centrifuged at 20,000 g for 5 min at 4° C. The supernatants were collected as whole cell extracts and stored at −80° C. Protein concentration was determined using Bicinchoninic Acid assay (Thermo Fisher Scientific). Equivalent amounts of whole cell extract were electrophoresed sodium dodecyl sulfate polyacrylamide gel electrophoresis gel. The proteins were then transferred to Immobilon-FL polyvinylidene difluoride membrane (EMD Millipore) and blocked with Odyssey blocking buffer (LI-COR; Lincoln, NE). The blots were incubated with primary antibodies in Odyssey blocking buffer supplemented with 0.2% Tween 20. After incubation with fluorescent dye-conjugated secondary antibodies, proteins of interest were detected using ChemiDoc MP imaging system (Bio-Rad). The primary and secondary antibodies used for Western blot analysis were: anti-KIAA0930 antibody (NBP2-84553) from Novus Biologicals (Centennial, CO); Alexa Fluor 647-conjugated antirabbit or antimouse IgG from Abcam (Boston, MA); Anti-ß-actin antibody from Thermo Fisher Scientific.

Measurement of C2C12 myotube atrophy in vitro. C2C12 myoblasts were seeded at 6.25×10⁴ cells/0.5 ml/well in a 24-well plate and cultured for 2 days. The medium was then switched to DME high-glucose medium supplemented with 2% HS (Differentiation medium; DM) and replenished after 1 and 3 days. Five-days after DM treatment, NCM or cancer cell CM were added at a final concentration of 10% in 0.25 ml/well of DM. Two-days after the treatment, cells were fixed with 3.7% formaldehyde in PBS for 15 min and blocked with 5% goat serum, 0.3% Triton X-100 in PBS for 1 hour at room temperature. Then the monolayer was incubated with anti-myosin heavy chain antibody (anti-MHC, from R&D Systems; Minneapolis, MN) overnight at 4° C., followed by Alexa Fluor 647 (AF647)-conjugated goat anti-mouse IgG antibody (Abcam; Boston, MA) and DAPI (Thermo Fisher Scientific) for nuclear staining. MHC/AF647-stained myotubes were photographed using Zeiss Observer Z1 (ZEISS; Pleasanton, CA) at ×10. The diameters were measured in a total of 40-50 myotubes from 4 random fields using ZEN Imaging Software (ZEISS).

Xenograft Experiment. Male NSG mice around 8 weeks of age were obtained from Jackson Laboratories. The mouse experiments were approved by the institutional animal care and use committee (IACUC) at City of Hope. PANC-1 at 5×10 6 cells were suspended in 100 μL of serum-free DME high-glucose medium with 50% matrigel and implanted orthotopically into the pancreas. For normal control, PBS was injected orthotopically. Mice were maintained up to 60 days after inoculation with body weight being measured at least once a week. At the time of euthanization, skeletal muscle, spleen, and pancreas with tumor were harvested, weighed, and snap frozen in liquid nitrogen, and stored at −80° C. Tibialis anterior (TA) was fixed with 10% formalin, embedded in paraffin, and cross-sections of TA was stained with hematoxylin and eosin (H-E). The images were captured using Zeiss Observer II (ZEISS). Cross section areas were measured using Image Pro Premier (Media Cybernetics, Rockville, Maryland).

Statistical Analyses. Results are expressed as means±SE. Statistical significance was determined by one way ANOVA, followed by Tukey's HSD (Honestly Significant Difference) test for data with three or more group, and chi-square test for comparison of muscle fiber cross section area distribution. Statistical analyses were performed with EZR. A value of P<0.05 was considered to be statistically significant.

Results

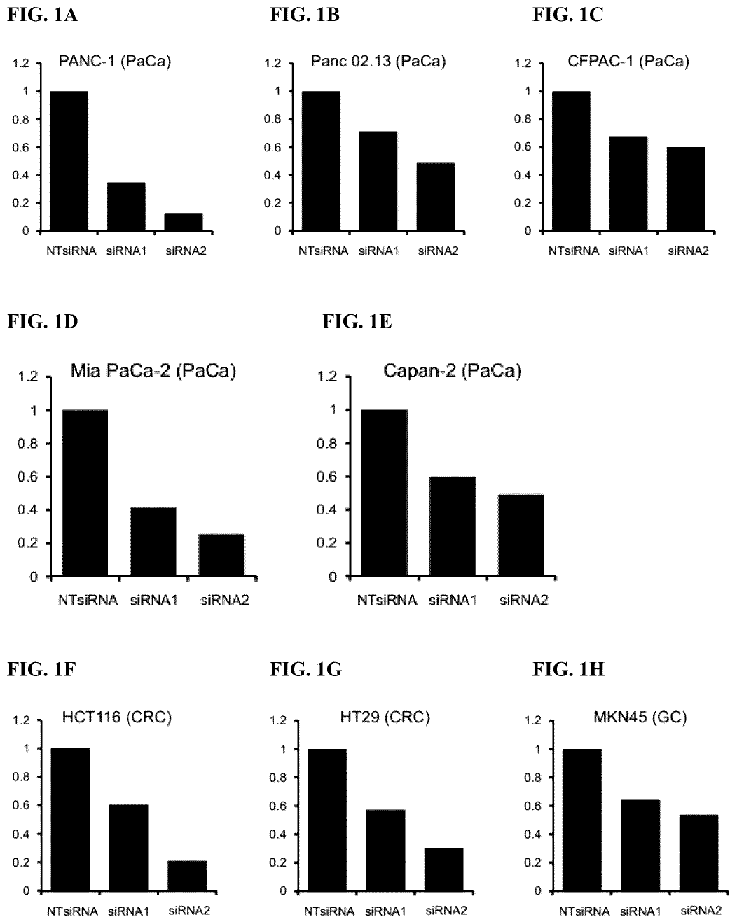

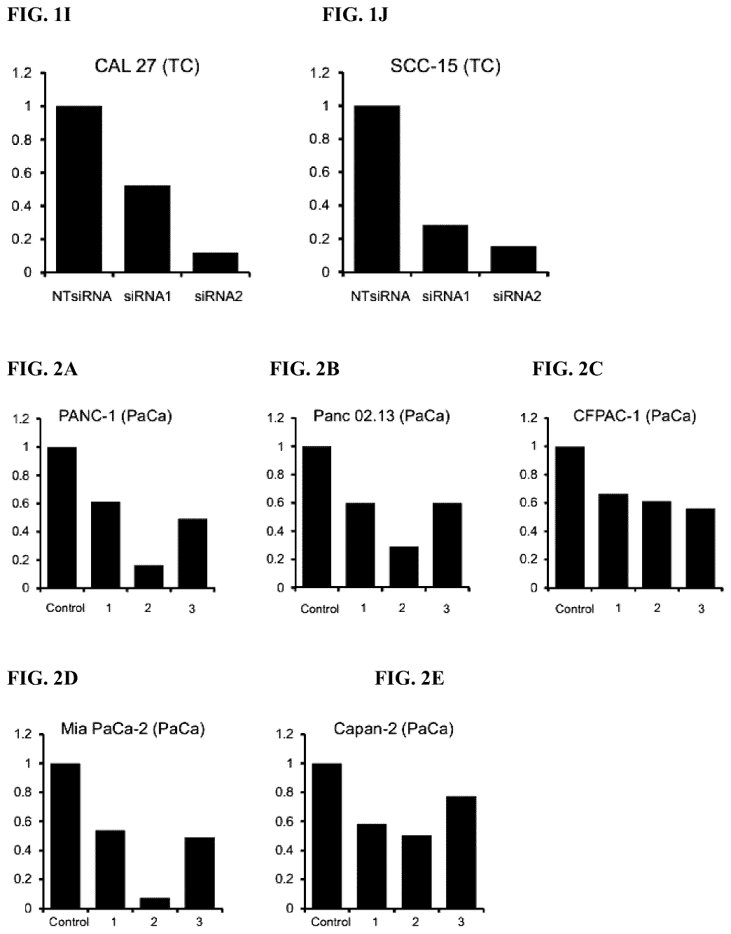

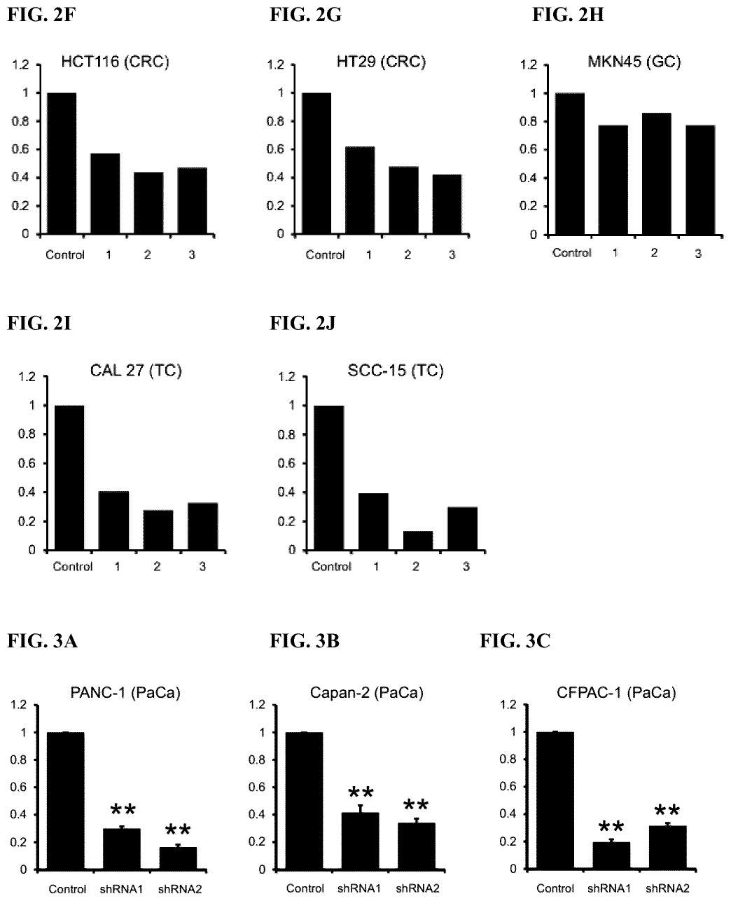

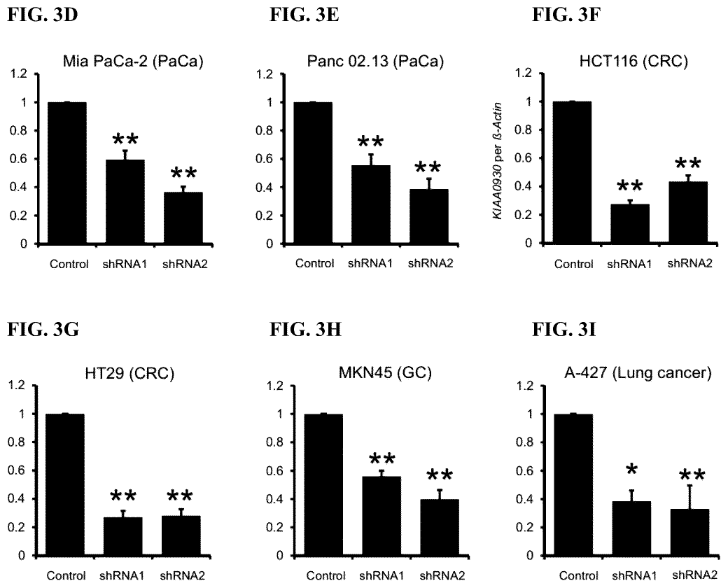

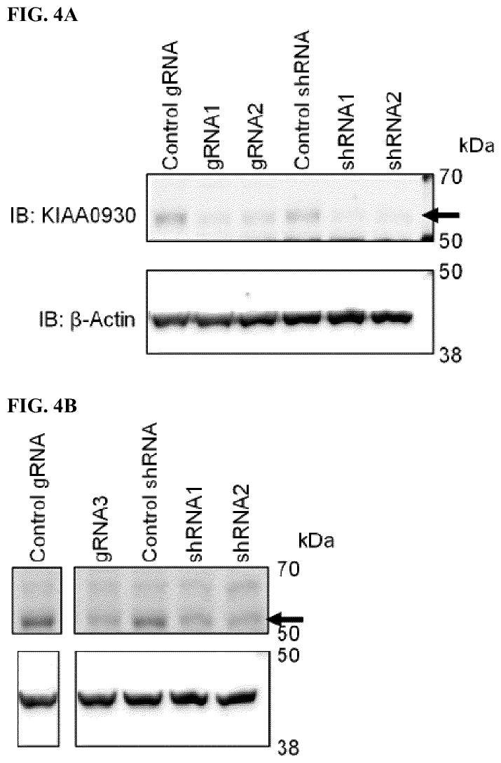

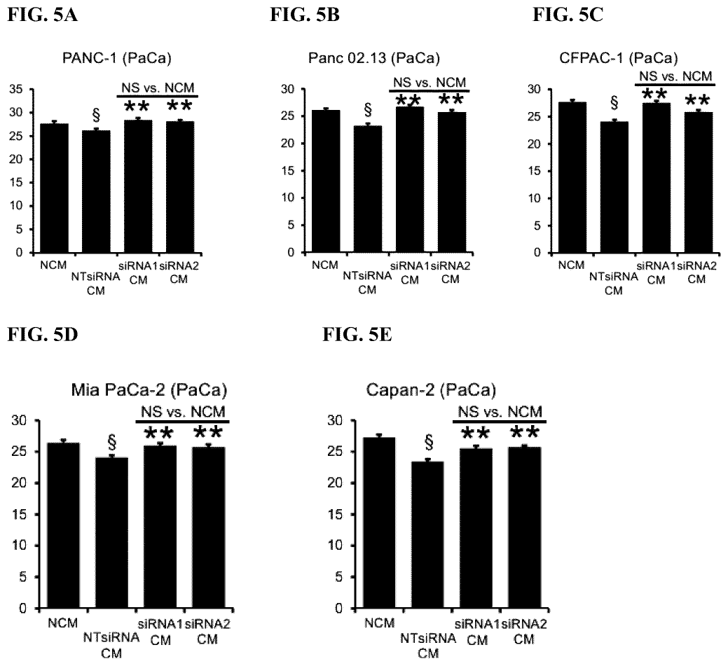

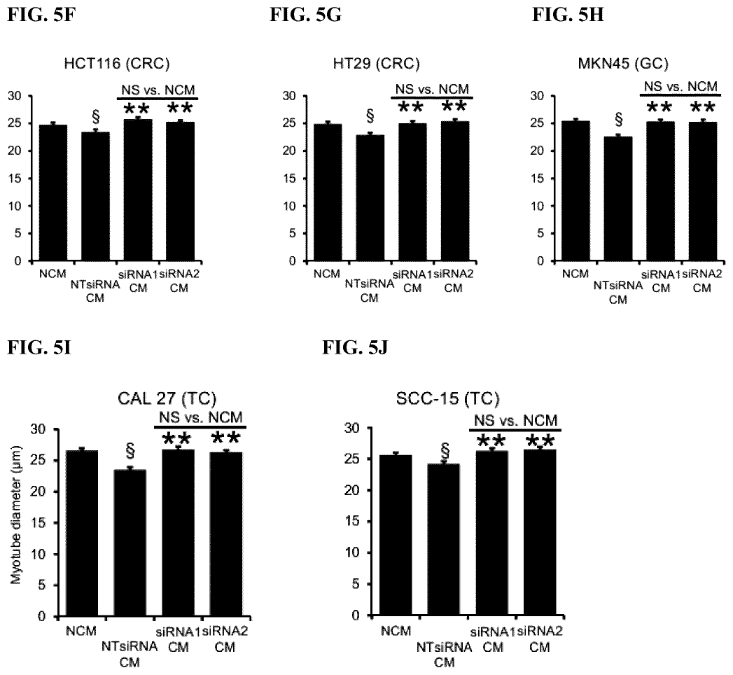

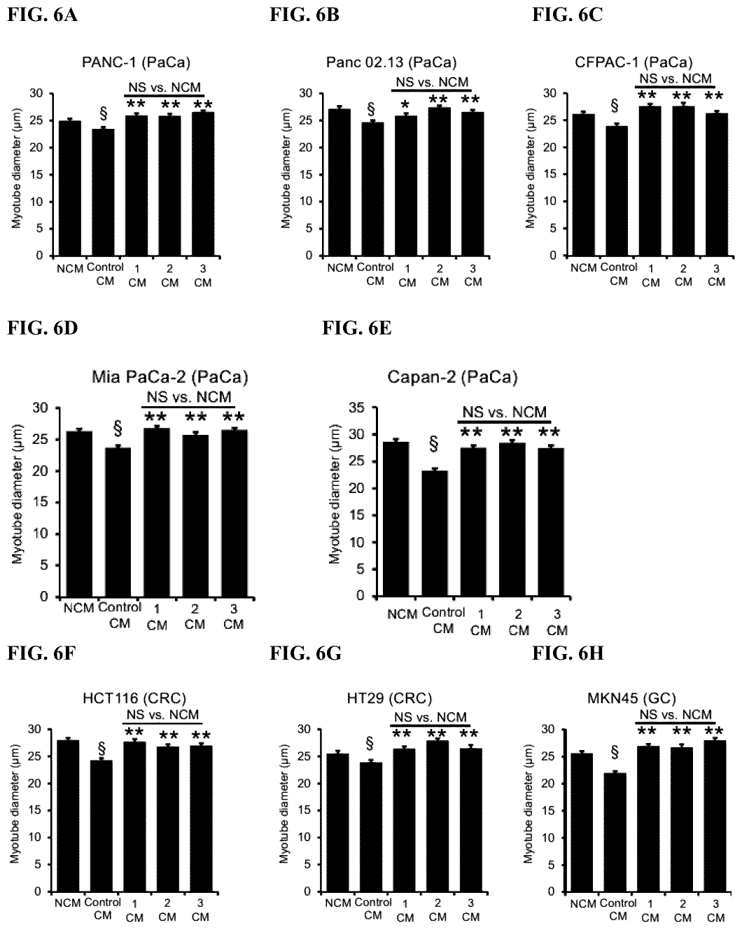

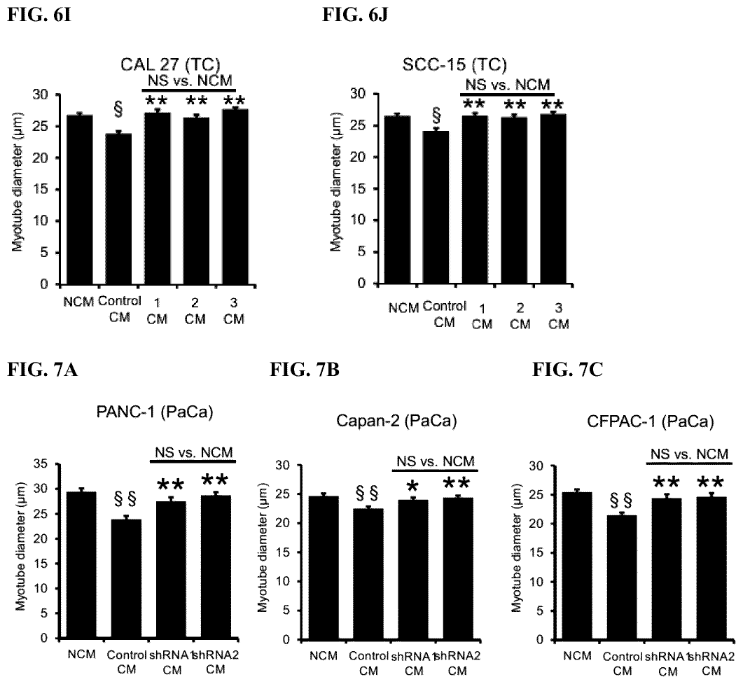

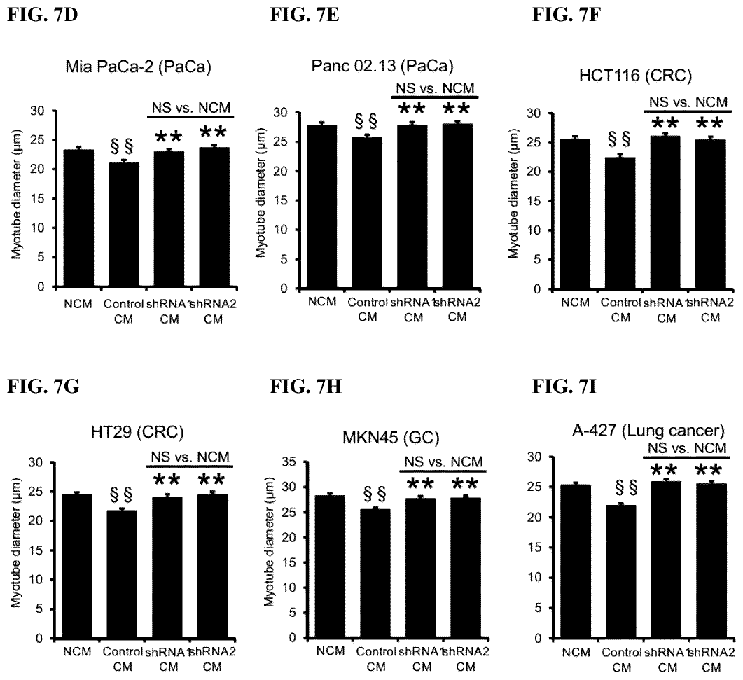

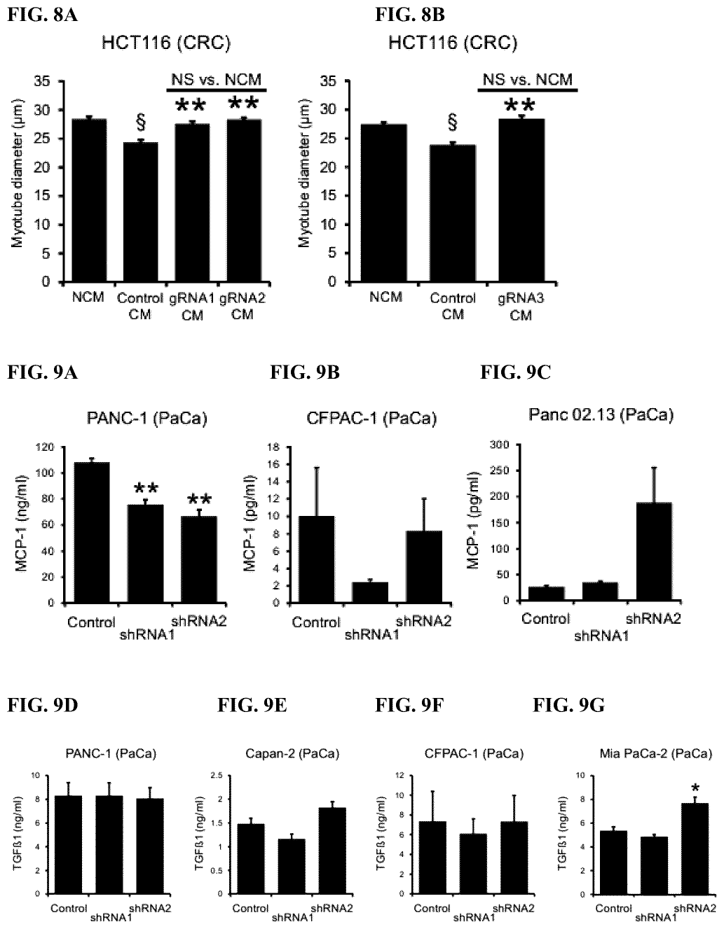

We examined whether suppression of KIAA0930 could affect cachexic phenotype in various cancer cells. KIAA0930 mRNA and protein were successfully reduced in cells by using siRNA, shRNA, GapmeR, and CRISPR-Cas9 methodologies (FIGS. 1-4). We then collected CM from these cells and add the CM to C2C12 myotubes. After 2 days, myotube diameter was measured. As shown in FIGS. 5-8, the diameter in myotubes treated with CM from control cancer cells we used in this study was shorter than NCM. However, CM from cells with low expression of KIAA0930 did not cause the decrease in diameter, showing that KIAA0930 suppression reduced cachexic phenotype in cancer cells. Importantly, this effect was consistently observed irrespective of cancer cell type and methods for suppression of KIAA0930.

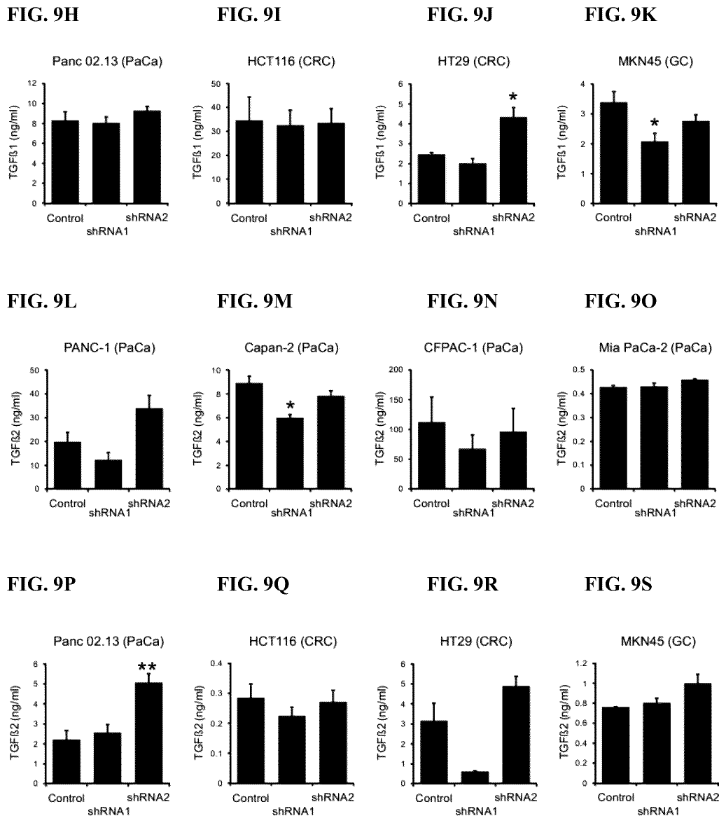

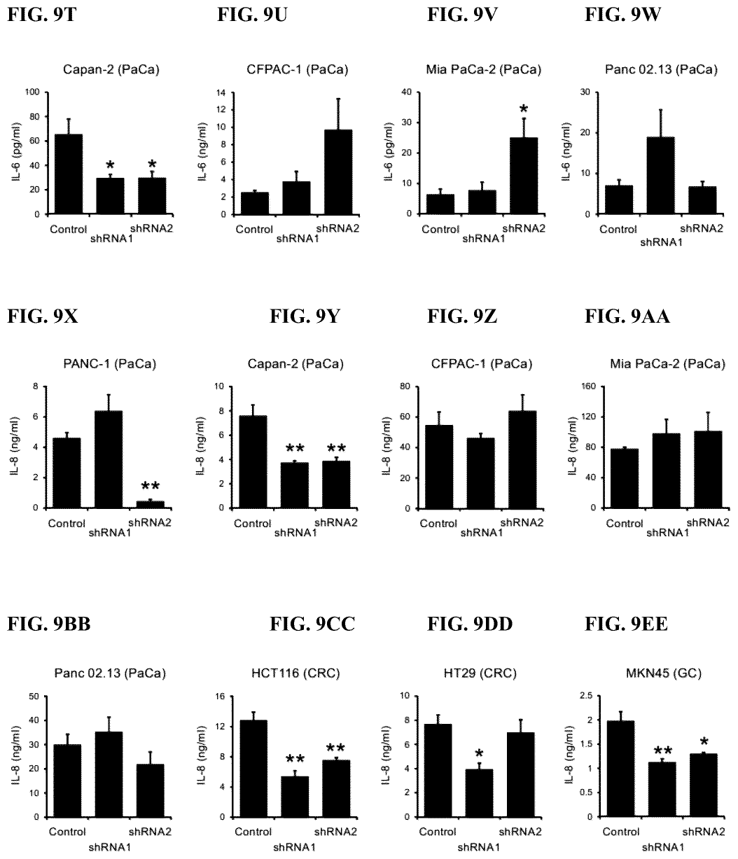

It has been reported that inflammatory cytokines/chemokines lead to muscle cachexia. Therefore, we examined several cytokine/chemokine amounts in CM from shRNA1, shRNA2-expressing cells. Knockdown of KIAA0930 did not exhibit consistent changes in cytokine levels, together with huge variations among cell lines (FIG. 9). These data indicate that the effect of KIAA0930 on cachexia is not due to changes in these cytokines.

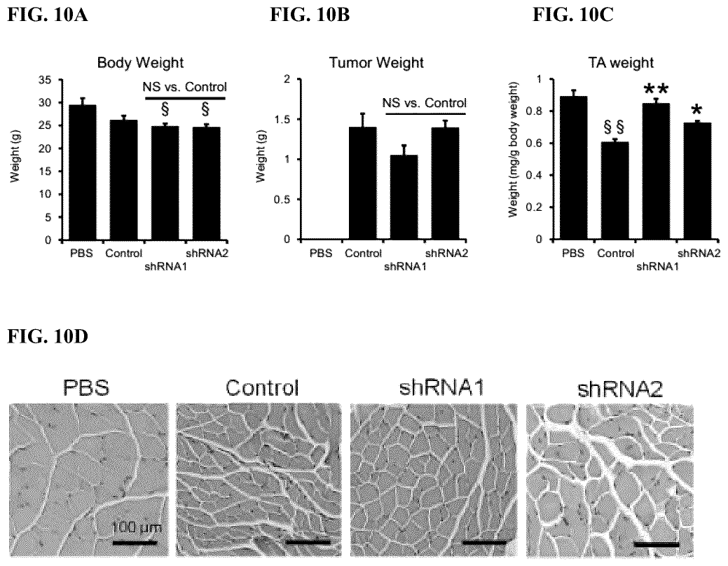

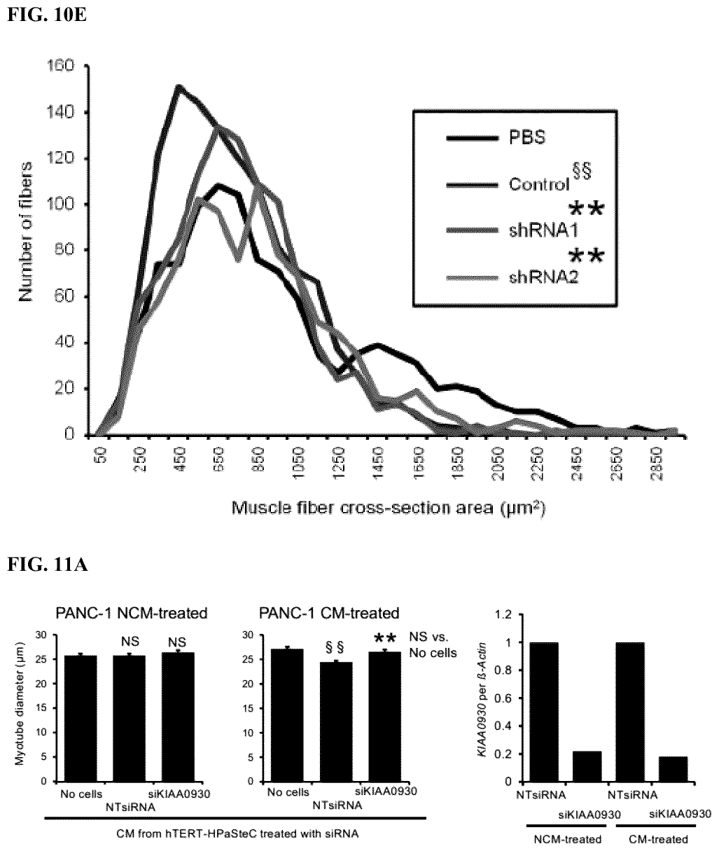

We next examined whether KIAA0930 knockdown ameliorated muscle atrophy in an orthotopic xenograft model. PANC-1 cells expressing shRNA1, shRNA2, or control were inoculated into pancreas in NSG mouse, and tumor-bearing mice were maintained for 8 weeks. Inoculation of control PANC-1 clearly decreased TA weight compared to PBS injection group, indicating that cachexia is induced in this orthotopic model (FIGS. 10A-10C). Although tumor weight in KIAA0930 knockdown PANC-1-bearing mice did not differ from control, TA weight was significantly higher than control. In agreement with this, the quantification of cross section areas in TA muscle revealed that PANC-1 cells expressing less KIAA0930 exhibited lower muscle atrophy (FIGS. 10D-10E).

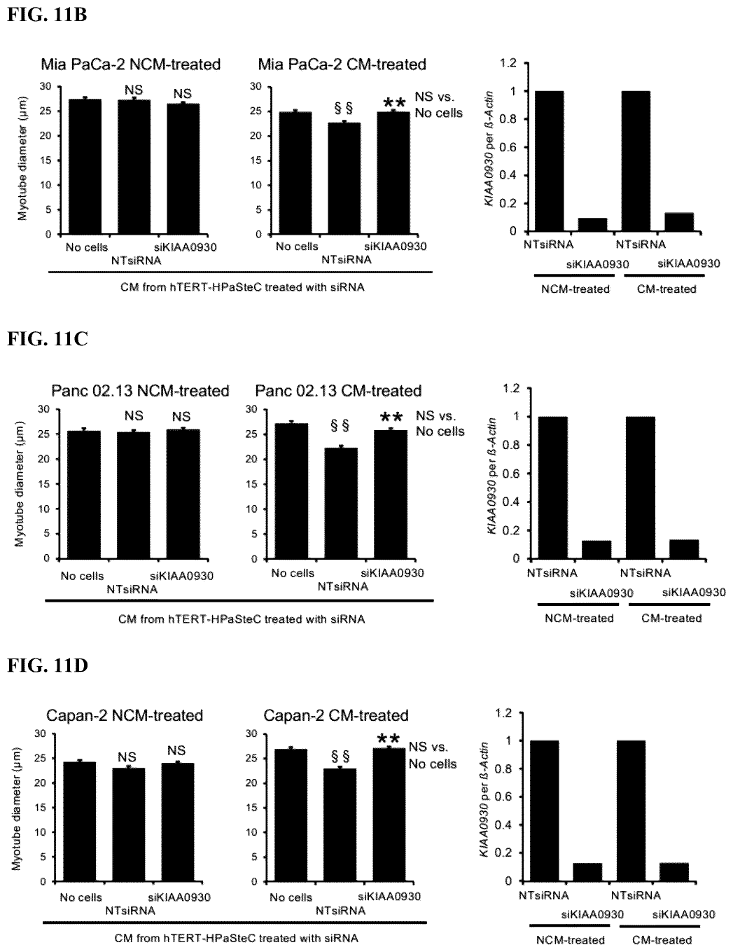

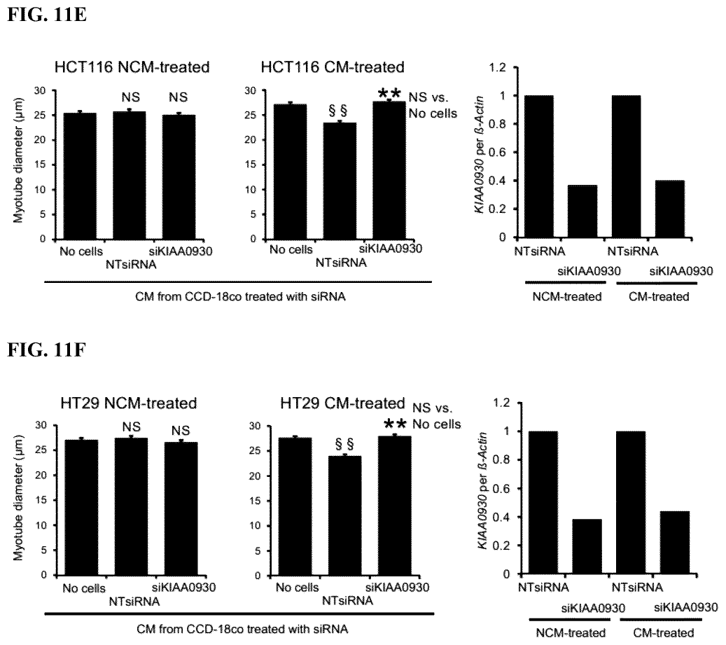

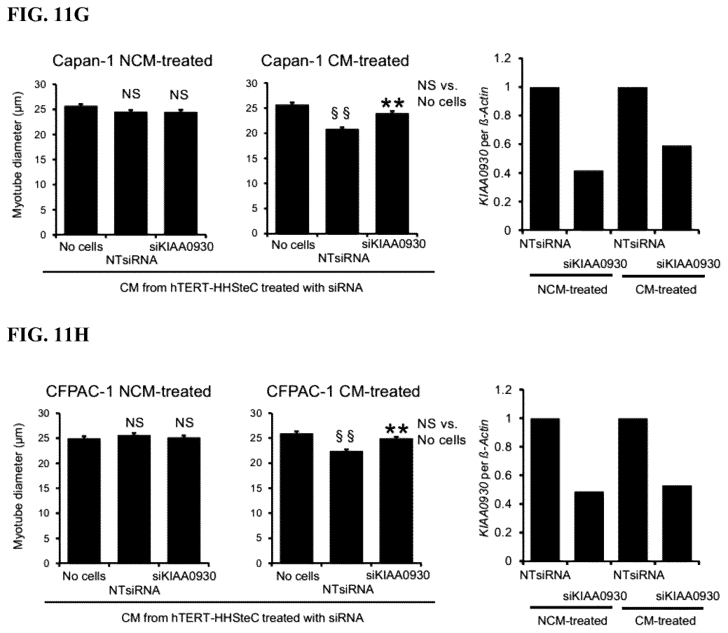

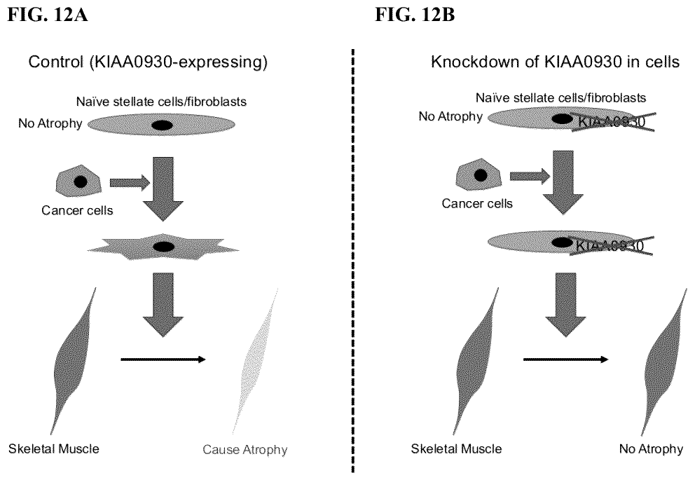

Cancer cells dynamically interact with stromal fibroblasts, endothelial cells and immune cells like microglia, macrophages, and lymphocytes, called tumor microenvironment (TME). TME increases multidrug resistance, cancer progression, and metastasis in part by stimulation of cytokine secretion. Baghban et al, Cell Commun Signal. 2020; 18(1):59. In fact, it has been shown that primary pancreatic cancer cells influenced stromal cells to induce the secretion of IL-6 and IL-8 in vitro. Callaway et al, Cancers (Basel). 2019; 11(12). We therefore tested a possibility that cancer cells affect their microenvironment to confer cachexic phenotype. Pancreatic stellate cells (hTERT-HPaSteC) and colon fibroblasts (CCD-18co) were cultured with 10% CM from pancreatic cancer and colon cancer cells, respectively, and muscle atrophy experiments were carried out. KIAA0930 mRNA was suppressed in siKIAA0930-treated cells (FIGS. 11A-1111, right). NCM-treated hTERT-HPaSteC and CCD-18co did not show muscle atrophic phenotype even though KIAA0930 mRNA was knocked down (FIGS. 11A-11F, left). CM from PANC-1, Mia PaCa-2, Panc 02.13, Capan-2, HCT116, and HT29-treated cells reduced myotube diameter (FIGS. 11A-11F, middle; compare left two columns), however, the reduction was inhibited when KIAA0930 was knocked down (FIGS. 11A-11F, middle; compare right two columns). Since pancreatic cancer metastasizes to liver, we treated hepatic stellate cells with CM from either Capan-1 or CFPAC-1 cells, which are obtained from a liver metastasis, and tested myotube atrophy. In agreement with FIGS. 11A-11F, cancer CM induced muscle atrophic phenotype in hTERT-HHSteC, and knockdown of KIAA0930 in the cells inhibited the phenotype (FIGS. 11G-1111). These data show that the inhibitory function of KIAA0930 suppression on cachexic phenotype is observed in not only cancer cells, but also surrounding cells, such as stellate cells and fibroblasts (FIG. 12).

Thus, we show that suppression of KIAA0930 in both cancer cells and surrounding cells leads to less cachexic phenotype both in vitro and in vivo, and that inhibition of KIAA0930 is a novel target for cachexia.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of preventing or treating a wasting syndrome in a subject in need thereof, the method comprising administering to the subject an effective amount of a KIAA0930 inhibitor.

2. The method of claim 1, wherein the KIAA0930 inhibitor is a short-hairpin RNA, a small interference RNA, a piwi-interacting RNA, a microRNA, a CRISPR Cas guide RNA, an antisense oligonucleotide, or a small molecule compound.

3. The method of claim 1, wherein the KIAA0930 inhibitor is:
(a) a short-hairpin RNA comprising SEQ ID NO:8;
(b) a short-hairpin RNA comprising SEQ ID NO:9;
(c) a small-interference RNA comprising SEQ ID NO:1;
(d) a small-interference RNA comprising SEQ ID NO:2;
(e) a GapmeR comprising SEQ ID NO:3;
(f) a GapmeR comprising SEQ ID NO:4;
(g) a GapmeR comprising SEQ ID NO:5;
(h) a CRISPR Cas9 guide RNA comprising SEQ ID NO:6;
(i) a CRISPR Cas9 guide RNA comprising SEQ ID NO:7; or
(j) a CRISPR Cas9 guide RNA comprising SEQ ID NO:10.

4. The method of claim 1, wherein the KIAA0930 inhibitor is a GapmeR.

5. The method of claim 1, wherein the KIAA0930 inhibitor is a CRISPR Cas9 guide RNA.

6. The method of claim 1, wherein the KIAA0930 inhibitor is a morpholinooligonucleotide.

7. The method of claim 1, wherein the KIAA0930 inhibitor is a CRISPR Cas 12 guide RNA.

8. The method of claim 1, wherein the wasting syndrome is cancer cachexia.

9. The method of claim 8, wherein the cancer is pancreatic cancer, colorectal cancer, gastric cancer, head and neck cancer, or lung cancer.

10. The method of claim 1, wherein the wasting syndrome is weight loss, fat loss, muscle atrophy, anorexia, asthenia, or anemia.

11. The method of claim 1, wherein the wasting syndrome is muscle atrophy.

12. The method of claim 1, wherein the KIAA0930 inhibitor reduces or inhibits fat and muscle loss; reduces or inhibits muscle atrophy; reduces or inhibits anorexia; reduces or inhibits asthenia; reduces or inhibits anemia; or a combination of two or more thereof.

13. The method of claim 1, wherein administering is oral, lingual, sublingual, parenteral, rectal, topical, transdermal, or pulmonary.

14. The method of claim 1, further comprising administering to the subject an effective amount of an anti-cancer agent.

15. A pharmaceutical composition comprising a KIAA0930 inhibitor and a pharmaceutically acceptable excipient.

16. The pharmaceutical composition of claim 15, wherein the KIAA0930 inhibitor is a short-hairpin RNA, a small interference RNA, a piwi-interacting RNA, a microRNA, a CRISPR Cas guide RNA, an antisense oligonucleotide, or a small molecule compound.

17. The pharmaceutical composition of claim 15, wherein the KIAA0930 inhibitor is:
(a) a short-hairpin RNA comprising SEQ ID NO:8;
(b) a short-hairpin RNA comprising SEQ ID NO:9;
(c) a small-interference RNA comprising SEQ ID NO:1;
(d) a small-interference RNA comprising SEQ ID NO:2;
(e) a GapmeR comprising SEQ ID NO:3;
(f) a GapmeR comprising SEQ ID NO:4;
(g) a GapmeR comprising SEQ ID NO:5;
(h) a CRISPR Cas9 guide RNA comprising SEQ ID NO:6;
(i) a CRISPR Cas9 guide RNA comprising SEQ ID NO:7; or
(j) a CRISPR Cas9 guide RNA comprising SEQ ID NO:10.

18. The pharmaceutical composition of claim 15, wherein the KIAA0930 inhibitor is a GapmeR or a morpholinooligonucleotide.

19. The pharmaceutical composition of claim 15, wherein the KIAA0930 inhibitor is CRISPR Cas9 guide RNA or a CRISPR Cas 12 guide RNA.

20. A KIAA0930 inhibitor, wherein the KIAA0930 inhibitor is:
(a) a short-hairpin RNA comprising SEQ ID NO:8;
(b) a short-hairpin RNA comprising SEQ ID NO:9;
(c) a small-interference RNA comprising SEQ ID NO:1;
(d) a small-interference RNA comprising SEQ ID NO:2;
(e) a GapmeR comprising SEQ ID NO:3;
(f) a GapmeR comprising SEQ ID NO:4;
(g) a GapmeR comprising SEQ ID NO:5;
(h) a CRISPR Cas9 guide RNA comprising SEQ ID NO:6;
(i) a CRISPR Cas9 guide RNA comprising SEQ ID NO:7; or
(j) a CRISPR Cas9 guide RNA comprising SEQ ID NO:10.
